(19) United States
(12) Patent Application Publication
(10) Pub. No.: US 2011/0008404 A1
(43) Pub. Date: Jan. 13, 2011

(54) Modification Of Biomaterials With Microgel Films

(75) Inventors: Louis Andrew Lyon, Marietta, GA (US); Andres J. Garcia, Atlanta, GA (US); Julia E. Babensee, Atlanta, GA (US)

Correspondence Address: TROUTMAN SANDERS LLP;5200 BANK OF AMERICA PLAZA, 600 PEACHTREE STREET, N.E., SUITE 5200, ATLANTA, GA, 30308-2216, US

(73) Assignee: Georgia Tech Research Corporation, Atlanta, GA (US)

(21) Appl. No.: 12/809,433

(22) PCT Filed: Dec. 19, 2008
(86) PCT No.: PCT/US08/87786

§ 371 Date: Sep. 27, 2010

Related U.S. Application Data
(62) Division of application No. 61/014,972, filed on Dec. 19, 2007.

(51) Int. Cl. A61K 9/00; A61K 47/30; A61P 29/00; A61P 17/02; H05H 1/00

(52) U.S. Cl. 424/422; 514/772.6; 427/569

(57) ABSTRACT

The various embodiments of the present disclosure relate generally to the modification of biomaterials with microgel films. More particularly, the various embodiments of the present invention are directed to the modification of biomaterials and medical devices with microgel thin films to alter a host's response to an implanted biomaterial or medical device. An embodiment of the present invention comprises a coated biomaterial comprising a non-fouling polymer film attached to at least a portion of a surface of the biomaterial, the non-fouling polymer film comprising a plurality of a cross-linked polymer microparticles, wherein at least a portion of the cross-linked polymer microparticles are covalently bonded to at least a portion of the surface of the biomaterial.

### RELATED APPLICATIONS

This application claims, under 35 U.S.C. §119(e), the benefit of U.S. Provisional Application Ser. No. 61/014,972, filed 19 Dec. 2007, the entire contents and substance of which are hereby incorporated by reference as if fully set forth below.

### STATEMENT OF FEDERALLY SPONSORED RESEARCH

This invention was made with U.S. Government support under Grant No. EEC-9731643 awarded by the National Science Foundation. The U.S. Government has certain rights in the invention.

### TECHNICAL FIELD

The various embodiments of the present disclosure relate generally to the modification of biomaterials with microgel films. More particularly, the various embodiments of the present invention are directed to the modification of biomaterials and medical devices with microgel thin films to alter a host's response to an implanted biomaterial or medical device.

### BACKGROUND OF THE INVENTION

Host inflammatory responses to implanted biomaterials limit device integration and biological performance for many classes of medical devices, including chemical biosensors, leads and electrodes for monitoring and/or stimulation, drug delivery systems, and orthopaedic implants, among others. These inflammatory responses to synthetic materials involve dynamic, multi-component, and inter-dependent reactions comprising biomolecule (e.g., protein) adsorption, leukocyte recruitment, adhesion, and activation, cytokine expression and release, macrophage fusion into multi-nucleated foreign body giant cells, tissue remodeling, and fibrous encapsulation. The duration and intensity of these stages are dependent upon the extent of injury created at the implantation site and the biomaterial physicochemical properties.

Significant biomaterial-based efforts have focused on engineering implant surface coatings to attenuate host inflammatory responses to implanted devices. Strategies focusing on the presentation or delivery of anti-inflammatory and/or pro-wound healing agents, such as heparin, dexamethasone, and superoxide dismutase mimetics, have demonstrated promising reductions in inflammatory responses and fibrous encapsulation. These approaches, however, are limited by complex delivery pharmacokinetics. In addition to these approaches, non-fouling (i.e. protein adsorption-resistant) coatings, including dense polymeric films and polymeric brushes have been pursued to modulate inflammatory responses to implanted materials. The rationale for these passive approaches is that reduction in protein adsorption will lead to reduced leukocyte adhesion and activation, thereby attenuating the extent of the foreign body reaction. Although many of these coatings exhibit reduced protein adsorption and leukocyte adhesion/activation in vitro, inconsistent results have been obtained regarding the ability of these materials to reduce in vivo acute and chronic inflammatory responses. Possible explanations for the mixed in vivo results with these coatings include insufficient non-fouling behavior, coating degradation, and inflammatory mechanism(s) independent from protein adsorption.

Hydrogels are three-dimensional networks of hydrophilic polymers, which have many applications in biomedicine and biotechnology due to their high water content, soft tissue-like consistency, and, potential biocompatibility. Hydrogels offer distinct advantages over traditional surface modifications, including high water content, high diffusivity for solute transport within polymer network, and the ability to incorporate multiple chemical functionalities to generate complex architectures. Accordingly, there is a need for micro-structured and nano-structured, non-fouling, hydrogel coatings for biomaterials to alter a host's response to an implanted material. It is to the provision of such non-fouling, hydrogel coatings for biomaterials that the various embodiments of the present invention are directed.

### SUMMARY

The various embodiments of the present disclosure relate generally to the modification of biomaterials with microgel films. More particularly, the various embodiments of the present invention are directed to the modification of biomaterials and medical devices with microgel films to alter a host's response to an implanted biomaterial or medical device.

Broadly described, an aspect of the present invention comprises a coated biomaterial comprising a non-fouling polymer film attached to at least a portion of a surface of the biomaterial, the non-fouling polymer film comprising a plurality of a cross-linked polymer microparticles, wherein at least a portion of the cross-linked polymer microparticles are covalently bonded to at least a portion of the surface of the biomaterial. In an embodiment of the present invention, the non-fouling polymer film adsorbs at least about 100% less protein than an uncoated biomaterial. In another embodiment of the present invention, the non-fouling polymer film adheres at least about 100% fewer cells than an uncoated biomaterial.

The non-fouling polymer film in its solvent swollen state comprises a thickness of about 10 nanometers to about 10 micrometers. In one embodiment of the present invention, the cross-linked polymer microparticles comprises poly(N-isopropylacrylamide) cross-linked with poly(ethylene glycol) diacrylate. More specifically, in an embodiment of the present invention, the poly(ethylene glycol) diacrylate has a molecular weight of less than about 575 Da and a concentration of about 2 mol %.

In various embodiments of the present invention, an uncoated biomaterial elicits a first bio-response when placed in a bio-environment, and the coated biomaterial comprising the non-fouling polymer film elicits a second bio-response that is different than the first bio-response when placed in a similar bio-environment. For example, the uncoated biomaterial elicits a first bio-response when placed in a bio-environment, and the coated biomaterial comprising the non-fouling polymer film elicits a second bio-response that is less than the first bio-response when placed a similar bio-environment. In such an example, the bio-environment is an in vivo system and the bio-response in an inflammatory response. In another example, the uncoated biomaterial elicits a first bio-response when placed in a bio-environment, and the coated biomaterial comprising the non-fouling polymer film elicits a second bio-response that is greater than the first bio-response when placed a similar bio-environment. In such an example, the bio-environment is an in vivo system and the bio-response in a wound healing response.

Another aspect of the present invention comprises a method for making a coated biomaterial comprising: providing a biomaterial having a surface; functionalizing at least a portion of the surface of the biomaterial; and covalently bonding a plurality of cross-linked polymer microparticles to at least a portion of the functionalized surface of the biomaterial. In an embodiment of the present invention, functionalizing at least a portion of the surface of the biomaterial comprises activating at least a portion of the surface of the biomaterial with a plasma, reacting the activated surface with oxygen to form a reactive species on the surface, grafting a linking moiety to the reactive species of the activated surface, and rendering the surface of the photoreactive with a photoaffinity labeling compound. In an embodiment of the present invention, covalently bonding a plurality of cross-linked polymer microparticles to at least a portion of the functionalized surface of the biomaterial to form a coated biomaterial comprises disposing a plurality of cross-linked polymer microparticles onto at least a portion of the functionalized surface of the biomaterial. In an embodiment of the present invention, covalently bonding a plurality of cross-linked polymer microparticles to at least a portion of the functionalized surface of the biomaterial to form a coated biomaterial further comprises reacting the photoreactive surface of the biomaterial with at least a portion of a plurality of cross-linked polymer microparticles in the presence of ultraviolet radiation.

In an embodiment of a method for making a coated biomaterial, an uncoated biomaterial elicits a first bio-response when placed in the bio-environment, and the coated biomaterial comprising the non-fouling polymer film elicits a second bio-response that is different than the first bio-response when placed a similar bio-environment. For example, the uncoated biomaterial elicits a first bio-response when placed in a bio-environment, and the coated biomaterial comprising the non-fouling polymer film elicits a second bio-response that is less than the first bio-response when placed a similar bio-environment. In such an example, the bio-environment is an in vivo system and the bio-response in an inflammatory response. In another example, the uncoated biomaterial elicits a first bio-response when placed in a bio-environment, and the coated biomaterial comprising the non-fouling polymer film elicits a second bio-response that is greater than the first bio-response when placed a similar bio-environment. In this example, the bio-environment is an in vivo system and the bio-response in a wound healing response.

An aspect of the present invention comprises a coated biomaterial capable of altering a bio-response, the biomaterial comprising a non-fouling polymer film attached to at least a portion of a surface of the biomaterial, the non-fouling polymer film comprising a plurality of a cross-linked polymer microparticles, wherein at least a portion of the cross-linked polymer microparticles are covalently bonded to at least a portion of the surface of the biomaterial, wherein an uncoated biomaterial elicits a first bio-response when placed in a bio-environment, and the coated biomaterial comprising the non-fouling polymer film elicits a second bio-response that is different than the first bio-response when placed in a similar bio-environment.

In one embodiment of the present invention, the uncoated biomaterial elicits a first bio-response when placed in a bio-environment, and the coated biomaterial comprising the non-fouling polymer film elicits a second bio-response that is less than the first bio-response when placed a similar the bio-environment. In such an embodiment, the bio-environment is an in vivo system and the bio-response in an inflammatory response. In another embodiment of the present invention, the uncoated biomaterial elicits a first bio-response when placed in a bio-environment, and the coated biomaterial comprising the non-fouling polymer film elicits a second bio-response that is greater than the first bio-response when placed a similar the bio-environment. In such an embodiment, the bio-environment is an in vivo system and the bio-response in a wound healing response.

In an embodiment of the present invention, the non-fouling polymer film adsorbs at least about 100% less protein than an uncoated biomaterial. In another embodiment of the present invention, the non-fouling polymer film adheres at least about 100% fewer cells than an uncoated biomaterial. The non-fouling polymer film in its solvent swollen state can comprises a thickness of about 10 nanometers to about 10 micrometers. In one embodiment of the present invention, the cross-linked polymer microparticles comprises poly(N-isopropylacrylamide) cross-linked with poly(ethylene glycol)diacrylate. More specifically, the poly(ethylene glycol)diacrylate has a molecular weight of less than about 575 Da and a concentration of about 2 mol %.

Another aspect of the present invention comprises a method for making a coated biomaterial comprising: providing a biomaterial having a surface; functionalizing at least a portion of the surface of the biomaterial; covalently bonding a plurality of cross-linked polymer microparticles to at least a portion of the functionalized surface of the biomaterial to form a coated biomaterial; and exposing the coated biomaterial to a bio-environment, wherein an uncoated biomaterial elicits a first bio-response when placed in the bio-environment, and the coated biomaterial comprising the non-fouling polymer film elicits a second bio-response that is different than the first bio-response when placed a similar bio-environment.

In an embodiment of the present invention, functionalizing at least a portion of the surface of the biomaterial comprises activating at least a portion of the surface of the biomaterial with a plasma, reacting the activated surface with oxygen to form a reactive species on the surface, grafting a linking moiety to the reactive species of the activated surface, and rendering the surface of the photoreactive with a photoaffinity labeling compound. In an embodiment of the present invention, covalently bonding a plurality of cross-linked polymer microparticles to at least a portion of the functionalized surface of the biomaterial to form a coated biomaterial comprises disposing a plurality of cross-linked polymer microparticles onto at least a portion of the functionalized surface of the biomaterial. In an embodiment of the present invention, covalently bonding a plurality of cross-linked polymer microparticles to at least a portion of the functionalized surface of the biomaterial to form a coated biomaterial further comprises reacting the photoreactive surface of the biomaterial with at least a portion of a plurality of cross-linked polymer microparticles in the presence of ultraviolet radiation.

In one embodiment of the present invention, the uncoated biomaterial elicits a first bio-response when placed in a bio-environment, and the coated biomaterial comprising the non-fouling polymer film elicits a second bio-response that is less than the first bio-response when placed a similar bio-environment. In such an embodiment, the bio-environment is an in vivo system and the bio-response in an inflammatory response. In an alternative embodiment of the present invention, the uncoated biomaterial elicits a first bio-response when placed in a bio-environment, and the coated biomaterial comprising the non-fouling polymer film elicits a second bio-response that is greater than the first bio-response when placed a similar bio-environment. In such an embodiment, the bio-environment is an in vivo system and the bio-response in a wound healing response.

Another aspect of the present invention comprises a method for altering a bio-response comprising: providing a coated biomaterial comprising a non-fouling polymer film attached to at least a portion of a surface of the biomaterial; exposing the coated biomaterial to a bio-environment; and eliciting a bio-response to the coated biomaterial, wherein an uncoated biomaterial elicits a first bio-response when placed in the bio-environment, and the coated biomaterial comprising the non-fouling polymer film elicits a second bio-response that is different than the first bio-response when placed in the bio-environment. In one embodiment, the uncoated biomaterial elicits a first bio-response when placed in a bio-environment, and the coated biomaterial comprising the non-fouling polymer film elicits a second bio-response that is less than the first bio-response when placed a similar bio-environment. In such an embodiment, the bio-environment is an in vivo system and the bio-response in an inflammatory response. In another embodiment of the present invention, the uncoated biomaterial elicits a first bio-response when placed in a bio-environment, and the coated biomaterial comprising the non-fouling polymer film elicits a second bio-response that is greater than the first bio-response when placed a similar bio-environment. In such an embodiment, the bio-environment is an in vivo system and the bio-response in a wound healing response.

An aspect of the present invention comprises a biomaterial comprising a non-fouling polymer film attached to at least a portion of a surface of the biomaterial, the non-fouling polymer film comprising an active agent and plurality of a cross-linked polymer microparticles, wherein at least a portion of the cross-linked polymer microparticles are covalently bonded to at least a portion of the surface of the biomaterial. In an embodiment of the present invention, the non-fouling polymer film adsorbs at least about 100% less protein than an uncoated biomaterial. In an embodiment of the present invention, the non-fouling polymer film adheres at least about 100% fewer cells than an uncoated biomaterial. In one embodiment of the present invention, the non-fouling polymer film in its solvent swollen state comprises a thickness of about 10 nanometers to about 10 micrometers. In an exemplary embodiment of the present invention, the cross-linked polymer microparticles comprises poly(N-isopropylacrylamide) cross-linked with poly(ethylene glycol)diacrylate. In an embodiment of the present invention, the poly(ethylene glycol)diacrylate has a molecular weight of less than about 575 Da and a concentration of about 2 mol %. In some embodiments of the present invention, the active agent comprises one or more active agents. In one embodiment of the present invention, the active agent is an anti-inflammatory agent.

In an embodiment of the present invention, the non-fouling polymer films provides an active agent to a bio-environment by display of an active agent on the surface of the non-fouling polymer film, passive diffusion of an active agent from the non-fouling polymer film, active delivery of the active agent from the non-fouling polymer film, or combinations thereof. In one embodiment of the present invention, the active agent is covalently associated with a cross-linked polymer microparticle by a stimulus responsive element, wherein a stimulus acts on the stimulus responsive element to release the active agent from the cross-linked polymer microparticle. In one embodiment of the present invention, the stimulus responsive element is a proteolytic cleavage site and the stimulus is a protease. In another embodiment of the present invention, the plurality of cross-linked polymer microparticles comprises a first population of microparticles comprising one or more active agents and a second population of microparticles comprising one or more active agent.

Another aspect of the present invention comprises a method for making a coated biomaterial comprising an active agent comprising: providing a biomaterial having a surface; functionalizing at least a portion of the surface of the biomaterial; covalently bonding at least a plurality of cross-linked polymer microparticles to at least a portion of the functionalized surface of the biomaterial to form a coated biomaterial; and providing an active agent to at least a portion of the non-fouling polymer film. In an embodiment of the present invention, functionalizing at least a portion of the surface of the biomaterial comprises activating at least a portion of the surface of the biomaterial with a plasma, reacting the activated surface with oxygen to form a reactive species on the surface, grafting a linking moiety to the reactive species of the activated surface, and rendering the surface of the photoreactive with a photoaffinity labeling compound. In an embodiment of the present invention, covalently bonding a plurality of cross-linked polymer microparticles to at least a portion of the functionalized surface of the biomaterial to form a coated biomaterial comprises disposing a plurality of cross-linked polymer microparticles onto at least a portion of the functionalized surface of the biomaterial. In an embodiment of the present invention, covalently bonding a plurality of cross-linked polymer microparticles to at least a portion of the functionalized surface of the biomaterial to form a coated biomaterial further comprises reacting the photoreactive surface of the biomaterial with at least a portion of a plurality of cross-linked polymer microparticles in the presence of ultraviolet radiation. In an embodiment of the present invention, providing an active agent to at least a portion of the non-fouling polymer film comprises providing one or more active agents to at least a portion of the non-fouling polymer. In an embodiment of the present invention, providing an active agent to at least a portion of the non-fouling polymer film comprises biofunctionalization of at least a portion of the plurality of cross-linked polymer microparticles with a chemoligation motif.

Another aspect of the present invention comprises a method for treating a bio-environment comprising: providing a coated biomaterial comprising a non-fouling polymer film attached to at least a port ion of a surface of the biomaterial, the non-fouling polymer film comprising an active agent; exposing the coated biomaterial to a bio-environment; and providing an active agent from the coated biomaterial to the bio-environment. In an embodiment of the present invention, the non-fouling polymer film in its solvent swollen state comprises a thickness of about 10 nanometers to about 10 micrometers. In an embodiment of the present invention, the cross-linked polymer microparticles comprises poly(N-isopropylacrylamide) cross-linked with poly(ethylene glycol)diacrylate. For example, in an embodiment of the present invention, the poly(ethylene glycol)diacrylate has a molecular weight of less than about 575 Da and a concentration of about 2 mol %.

In an embodiment of the present invention, the active agent comprises one or more active agents. For example, in an embodiment of the present invention, the active agent is an anti-inflammatory agent and the bio-environment is an in vivo system. In an embodiment of the present invention, providing an active agent from the coated biomaterial to the bio-environment comprises displaying an active agent on the surface of the non-fouling polymer film, passively diffusing an active agent from the non-fouling polymer film to the bio-environment, actively delivering an active agent from the non-fouling polymer film to the bio-environment, or combinations thereof. In another embodiment of the present invention, actively delivering an active agent from the non-fouling polymer film to the bio-environment comprises actively delivering an active agent from the non-fouling polymer film in response to a stimulus. In an embodiment of the present invention, the stimulus is a protease or an enzyme.

An aspect of the present invention comprises a coated non-PET biomaterial comprising a non-fouling polymer film attached to at least a portion of a surface of the non-PET biomaterial, the non-fouling polymer film comprising a plurality of a cross-linked polymer microparticles, wherein at least a portion of the cross-linked polymer microparticles are covalently bonded to at least a portion of the surface of the non-PET biomaterial. In an embodiment of the present invention, an uncoated non-PET biomaterial elicits a first bio-response when placed in a bio-environment, and the coated non-PET biomaterial comprising the non-fouling polymer film elicits a second bio-response that is different than the first bio-response when placed in a similar bio-environment. In one embodiment, the uncoated non-PET biomaterial elicits a first bio-response when placed in a bio-environment, and the coated non-PET biomaterial comprising the non-fouling polymer film elicits a second bio-response that is less than the first bio-response when placed a similar bio-environment. In such an embodiment, the bio-environment is an in vivo system and the bio-response in an inflammatory response. In another embodiment, the uncoated non-PET biomaterial elicits a first bio-response when placed in a bio-environment, and the coated non-PET biomaterial comprising the non-fouling polymer film elicits a second bio-response that is greater than the first bio-response when placed a similar the bio-environment. In such an embodiment, the bio-environment is an in vivo system and the bio-response in a wound healing response.

In an embodiment of the present invention, the non-fouling polymer film adsorbs at least about 100% less protein than an uncoated biomaterial. In an embodiment of the present invention, the non-fouling polymer film adheres at least about 100% fewer cells than an uncoated biomaterial. In an embodiment of the present invention, the non-fouling polymer film in its solvent swollen state comprises a thickness of about 10 nanometers to about 10 micrometers. In one embodiment, the cross-linked polymer microparticles comprises poly(N-isopropylacrylamide) cross-linked with poly(ethylene glycol)diacrylate. For example, in an embodiment of the present invention, the poly(ethylene glycol)diacrylate has a molecular weight of less than about 575 Da and a concentration of about 2 mol %.

An aspect of the present invention comprises a method for making a coated non-PET biomaterial comprising: providing a non-PET biomaterial having a surface; functionalizing at least a portion of the surface of the non-PET biomaterial; and covalently bonding a plurality of cross-linked polymer microparticles to at least a portion of the functionalized surface of the non-PET biomaterial. In an embodiment of the present invention, functionalizing at least a portion of the surface of the non-PET biomaterial comprises activating at least a portion of the surface of the non-PET biomaterial with a plasma, reacting the activated surface with oxygen to form a reactive species on the surface, grafting a linking moiety to the reactive species of the activated surface, and rendering the surface of the photoreactive with a photoaffinity labeling compound. In an embodiment of the present invention, covalently bonding a plurality of cross-linked polymer microparticles to at least a portion of the functionalized surface of the non-PET biomaterial to form a coated biomaterial comprises disposing a plurality of cross-linked polymer microparticles onto at least a portion of the functionalized surface of the non-PET biomaterial. In an embodiment of the present invention, covalently bonding a plurality of cross-linked polymer microparticles to at least a portion of the functionalized surface of the non-PET biomaterial to form a coated biomaterial further comprises reacting the photoreactive surface of the non-PET biomaterial with at least a portion of a plurality of cross-linked polymer microparticles in the presence of ultraviolet radiation.

In an embodiment of the present invention, an uncoated non-PET biomaterial elicits a first bio-response when placed in the bio-environment, and the coated non-PET biomaterial comprising the non-fouling polymer film elicits a second bio-response that is different than the first bio-response when placed a similar bio-environment. In one embodiment of the present invention, the uncoated non-PET biomaterial elicits a first bio-response when placed in a bio-environment, and the coated non-PET biomaterial comprising the non-fouling polymer film elicits a second bio-response that is less than the first bio-response when placed a similar bio-environment. In such an embodiment, the bio-environment is an in vivo system and the bio-response in an inflammatory response. In another embodiment of the present invention, the uncoated non-PET biomaterial elicits a first bio-response when placed in a bio-environment, and the coated non-PET biomaterial comprising the non-fouling polymer film elicits a second bio-response that is greater than the first bio-response when placed a similar bio-environment. In such an embodiment, the bio-environment is an in vivo system and the bio-response in a wound healing response.

Another aspect of the present invention comprises a method for altering a bio-response comprising: providing a coated non-PET biomaterial comprising a non-fouling polymer film attached to at least a portion of a surface of the non-PET biomaterial; exposing the coated non-PET biomaterial to a bio-environment; and eliciting a bio-response to the coated non-PET biomaterial, wherein an uncoated biomaterial elicits a first bio-response when placed in the bio-environment, and the coated biomaterial comprising the non-fouling polymer film elicits a second bio-response that is different than the first bio-response when placed in a similar bio-environment. In one embodiment, the uncoated non-PET biomaterial elicits a first bio-response when placed in a bio-environment, and the coated non-PET biomaterial comprising the non-fouling polymer film elicits a second bio-response that is less than the first bio-response when placed a similar bio-environment. In such an embodiment, the bio-environment is an in vivo system and the bio-response in an inflammatory response. In another embodiment of the present invention, the uncoated non-PET biomaterial elicits a first bio-response when placed in a bio-environment, and the coated non-PET biomaterial comprising the non-fouling polymer film elicits a second bio-response that is greater than the first bio-response when placed a similar bio-environment. In such an embodiment, the bio-environment is an in vivo system and the bio-response in a wound healing response.

Other aspects and features of embodiments of the present invention will become apparent to those of ordinary skill in the art, upon reviewing the following description of specific, exemplary embodiments of the present invention in conjunction with the accompanying figures.

### BRIEF DESCRIPTION OF DRAWINGS

FIG. 1 is a schematic of a strategy for covalent tethering of microgels onto a poly(ethylene terephthalate) surface.

FIG. 2 is a schematic of a dynamic microgel-based coating.

FIG. 3 illustrates absorption spectra for desorbed Toluidine Blue O dye from bare PET and poly(acrylic acid) grafted PET before and after modification with 4-aminobenzophenone.

FIGS. 4a-c are a 3D rendering of AFM images for (a) bare PET and (b and c) microgel-modified PET.

FIGS. 5a-b are a 3D rendering of AFM image of microgels spin coated onto pAAc-grafted PET (a) without benzophenone modification and (b) with benzophenone modification but without UV irradiation.

FIGS. 6a-b demonstrate macrophage adhesion on (a) bare PET and (b) PET covalently functionalized by microgels. Adherent cells were stained. (Scale bar 100 mm)

FIGS. 7a-g illustrate the surface characterization of biomaterials.

FIGS. 8a-b demonstrate the topography of biomaterial surfaces.

FIG. 9 provides protein adsorption profiles for biomaterial surfaces.

FIGS. 10a-d demonstrate murine IC-21 macrophage adhesion to biomaterial surfaces.

FIGS. 11a-d illustrate in vitro human primary macrophage adhesion to biomaterial surfaces.

FIGS. 12a-e illustrate in vivo leukocyte adhesion to implanted biomaterial surfaces.

FIGS. 13a-g demonstrate quantification of in vivo intracellular cytokine expression by flow cytometric analysis.

### DETAILED DESCRIPTION

Cell-material interactions regulate host responses to implanted devices and tissue-engineered constructs. Upon implantation, synthetic materials dynamically adsorb proteins and other biomolecules, which trigger non-specific inflammatory responses, culminating in a foreign body reaction and fibrous encapsulation of the implant. This fibrotic response limits device integration and biological performance in numerous biomedical applications, including pacemaker leads, neural electrodes, chemical biosensors, and orthopaedic implants, among others. Thus, non-specific inflammatory events associated with existing synthetic surfaces severely limit the in vivo performance of various implanted devices.

An aspect of the present invention comprises a coated biomaterial comprising a non-fouling polymer film attached to at least a portion of a surface of the biomaterial, the non-fouling polymer film comprising a plurality of a cross-linked polymer microparticles, wherein at least a portion of the cross-linked polymer microparticles are covalently bonded to at least a portion of the surface of the biomaterial.

As used herein, the term “biomaterial” refers to many materials, both natural and synthetic, used to replace part of a living system or to function in intimate contact with living tissue. Biomaterials are intended to interface with biological systems to evaluate, treat, augment, or replace a tissue, organ, or function of the body. Biomaterials can include, but are not limited to, ceramics, metals (e.g., Titanium), alloys, glasses, and polymers. In an exemplary embodiment, a biomaterial comprises a polymer, such as polyesters (e.g., poly(ethylene terephthalate) (PET)), polyacrylates (e.g., poly(methyl methacrylate) (PMMA)), silicone polymers, (e.g., polydimethylsiloxane (PDMS), silicone rubber), polyurethanes, and poly(lactides), among others.

The term “biomaterial” also comprises medical devices that can be made of ceramics, metals, alloys, glasses, and polymers, among others. Thus, the teachings of the present invention may be adapted for a variety of medical devices that may be used for embedding, insertion, contacting, implantation, or the like into a host including, but not limited to, biliary, urinary, or vascular stents; catheters; cannulas, or components thereof; plugs or fillers; coatings; constrictors; bone anchors (e.g., screws); bone grafts (e.g., plates and rods); bone cement; seeds or capsules; patches or dressings; dental implants; matrices for tissue engineering (e.g., sheets, tubes, plugs, and other macroscopic shapes); organs; skin; neural electrodes; pacemakers and the leads thereof; chemical biosensors (e.g., in-dwelling glucose sensors); prostheses (e.g., orthopaedic, mammary), joint replacements; heart valves; sutures; blood vessel prostheses; drug delivery devices (e.g., subcutaneous continuous release vehicles);among others. According to the various embodiments of the present invention, the biomaterials are suitable for in vitro and in vivo applications including, but not limited to use in a host, such as humans, animals, and plants.

As used herein, the term “coated” includes providing a polymer film to at least a portion of a surface of a biomaterial. Thus, a coated biomaterial, as defined herein, can comprise a biomaterial only having a portion of its surface coated by a polymer film. A coated biomaterial, as defined herein, can comprise a biomaterial having an entire surface or a substantially entire surface coated by the polymer film. Conversely, a person of ordinary skill in the art would realize that an “uncoated” biomaterial lacks a coating.

Various embodiments of the present invention comprise a non-fouling polymer film comprising a plurality of cross-linked polymer microparticles. As used herein, the term “non-fouling polymer film” includes polymer films exhibiting at least some resistance to protein adsorption. In an embodiment of the present invention, a non-fouling polymer film adsorbs at least about 100% less protein than an uncoated biomaterial. In another embodiment of the present invention, a non-fouling polymer film adsorbs at least about 250% less protein than an uncoated biomaterial. In yet another embodiment of the present invention, a non-fouling polymer film adsorbs at least about 500% less protein than an uncoated biomaterial. In still another embodiment of the present invention, a non-fouling polymer film adsorbs at least about 700% less protein than an uncoated biomaterial.

A non-fouling polymer film can also demonstrate some resistance to cell adhesion. In an embodiment of the present invention, a non-fouling polymer film adheres at least about 100% fewer cells than an uncoated biomaterial. In another embodiment of the present invention, a non-fouling polymer film adheres at least about 500% fewer cells than an uncoated biomaterial. In yet another embodiment of the present invention, a non-fouling polymer film adheres at least about 1,000% fewer cells than an uncoated biomaterial. In yet another embodiment of the present invention, a non-fouling polymer film adheres at least about 2,000% fewer cells than an uncoated biomaterial. In still another embodiment of the present invention, a non-fouling polymer film adheres at least about 4,000% fewer cells than an uncoated biomaterial.

A polymer film can have a variety of thicknesses. In an embodiment of the present invention, a polymer film in its solvent swollen form can have a thickness of about 10 nanometers to about 10 micrometers. In another embodiment of the present invention, a polymer film in its solvent swollen form can have a thickness of about 100 nanometers to about 1 micrometers. In an exemplary embodiment of the present invention, a polymer film in its solvent swollen form can have a thickness of about 300 nanometers.

A non-fouling polymer film comprises a plurality of cross-linked polymer microparticles. As used herein, the term “plurality” refers to more than one. A polymer microparticle can comprise many suitable hydrophilic polymers known in the art including, but not limited to, acrylates, acrylamides, acetates, acrylic acids, vinyl alcohols, glycols, polysaccharides, or combinations thereof. The cross-linker of the microparticles can be many suitable cross-linkers known in the art including, but not limited to, N,N′,methylenebis(acrylamide), poly(ethylene glycol) (PEG) diacrylate, N,N′-dihydroxyethylenebisacrylamide, N,O-(dimethacryloyl)hydroxylamine, ethylene glycol dimethacrylate, divinylbenzene, or combinations thereof. In various embodiments of the present invention, the polymer can have many topologies including, but not limited to, a branched topology, a graft topology, a comb topology, a star topology, a cyclic topology, a network topology, or combinations thereof, among others.

In an embodiment of the present invention, the polymer microparticle is a hydrogel microparticle (i.e., a microgel). In an exemplary embodiment of the present invention, the hydrogel microparticle comprises poly(N-isopropylacrylamide) (pNIPAm) cross-linked with a PEG diacrylate. In an embodiment of the present invention, PEG can have a molecular weight ranging from about 200 Da to less than about 2,000 Da. In an embodiment of the present invention, PEG can have a molecular weight of less than about 700 Da. In an exemplary embodiment of the present invention, PEG can have a molecular weight of about 575 Da. In an embodiment of the present invention, PEG can have a concentration ranging from about 0.2 mol % to about 20.0 mol %. In an exemplary embodiment of the present invention, PEG can be present at a concentration of about 2 mol %.

A polymer microparticle of the present invention can have many sizes. In an embodiment of the present invention, a polymer microparticle in solvent swollen form can have an average longest cross-sectional dimension of about 10 nanometers to about 5 micrometers. In an exemplary embodiment of the present invention, a polymer microparticle in solvent swollen form can have an average longest cross-sectional dimension of about 300 nanometers to about 600 nanometers. In an embodiment of the present invention, a polymer microparticle in solvent swollen form can have an average longest cross-sectional dimension of less than about 3 micrometers. In another embodiment of the present invention, a polymer microparticle in solvent swollen form can have an average longest cross-sectional dimension of less than about 600 nanometers. In another embodiment of the present invention, a polymer microparticle in solvent swollen form can have an average longest cross-sectional dimension of greater than about 300 nanometers. In another embodiment of the present invention, a polymer microparticle in solvent swollen form can have an average longest cross-sectional dimension of greater than about 50 nanometers.

In various embodiments of the present invention, at least a portion of the cross-linked polymer microparticles are covalently bonded to at least a portion of the surface of the biomaterial. The methods for covalently attaching a polymer microparticle to a biomaterial are quite diverse. A person of ordinary skill in the art would realize that the method of covalently attaching a polymer microparticle to a biomaterial depends largely on the chemical composition of the polymer microparticle and/or chemical composition of the biomaterial. For example, in the context of silicone-based biomaterials, a polymer microparticle can be covalently bonded to the silicone-based biomaterial through the use of silane chemistry. In another example, in the context of PET, a polymer microparticle can be covalently bonded to a PET-based biomaterial through the use of photoaffinity labeling compounds, such as benzophenones, aryl azide, and diazirines, among others. Photoaffinity labeling compounds can be used for polymer microparticles or biomaterials comprising functional groups including, but not limited to, phosphoryls, amines, acetates, carboxylates, aldehydes, hydrazides, sulfhydryls, hydroxyls, or ketones. In another example, in the context of metals, a polymer particle can be covalently attached to the metal surface through the use of strong chemisorption interactions, such as thiol attachment to gold and silver, or benzene diol attachment to titanium, among others.

An aspect of the present invention comprises a coated biomaterial capable of altering a bio-response, the biomaterial comprising a non-fouling polymer film attached to at least a portion of a surface of the biomaterial, the non-fouling polymer film comprising a plurality of a cross-linked polymer microparticles, wherein at least a portion of the cross-linked polymer microparticles are covalently bonded to at least a portion of the surface of the biomaterial, wherein an uncoated biomaterial elicits a first bio-response when placed in a bio-environment, and the coated biomaterial comprising the non-fouling polymer film elicits a second bio-response that is different than the first bio-response when placed in a similar bio-environment.

As used herein, the term “bio-environment” includes many biologically-based environments, including both in vitro and in vivo systems capable of providing a bio-response. A bio-environment can include a cell culture (e.g., eukaryotic, prokaryotic), a bioreactor, a tissue, an organ, or an organism (e.g., an animal, plant, human), among others. A bio-response can comprise many biological responses, activities, functions, or processes including, but not limited to adsorption of proteins and other biomolecules, cell adhesion, leukocyte activation, intracellular signaling, intercellular signaling, cytokine secretion, chemokine secretion, complement activation, inflammatory responses, production and/or release of pro-inflammatory effector molecule (e.g., reactive oxygen and nitrogen intermediates), fibrous encapsulation, receptor-ligand interactions, antigen-antibody interactions, cellular proliferation, cellular apoptosis, and cellular differentiation, among others.

According to various embodiments of the present invention, an uncoated biomaterial elicits a first bio-response when placed in a bio-environment, and the coated biomaterial comprising the non-fouling polymer film elicits a second bio-response that is different than the first bio-response when placed in a similar bio-environment. For example, the inflammatory response to an implanted uncoated biomaterial comprises a cascade of molecular and cellular events including biomolecule (e.g., protein) adsorption, leukocyte recruitment, adhesion and activation of leukocytes, cytokine expression and release, macrophage fusion into multi-nucleated foreign body giant cells, tissue remodeling, and fibrous encapsulation. Thus, according to the various embodiments of the present invention, the inflammatory response to the an implanted coated biomaterial would include reduced biomolecule (e.g., protein) adsorption, decreased leukocyte recruitment, reduced adhesion and activation of leukocytes, decreased pro-inflammatory cytokine expression and release, a reduction of macrophage fusion into multi-nucleated foreign body giant cells, and limited tissue remodeling and fibrous encapsulation. This example is not intended to suggest that coating of a biomaterial according to the embodiments of the present invention will always result in an inhibition or reduction of an undesired bio-response. In contrast, the biomaterials of the present invention can comprise bioactive interfaces or active agents capable of promoting or enhancing desired bio-responses (e.g., wound healing, cell proliferation, cell differentiation).

An aspect of the present invention comprises a method for making a coated biomaterial comprising: providing a biomaterial having a surface; functionalizing at least a portion of the surface of the biomaterial; covalently bonding a plurality of cross-linked polymer microparticles to at least a portion of the functionalized surface of the biomaterial.

Functionalizing at least a portion of the surface of the biomaterial can comprise many methods know in the art for the functionalization of a surface. Many biomaterials (e.g., PET) are inert and are not suitable for direct functionalization. Thus, functionalization of a biomaterial surface may comprise activation of at least a portion of the surface of the biomaterial and functionalizing at least a portion of activated the surface of the biomaterial. Various functionalities can be introduced onto the biomaterial surface including, but not limited to amine, carboxyl, peroxide, and hydroxyl moieties. In an exemplary embodiment of the present invention, functionalizing at least a portion of the surface of the biomaterial comprise chemical modification of the biomaterial surface with limited effects to the bulk/mechanical properties of the biomaterial.

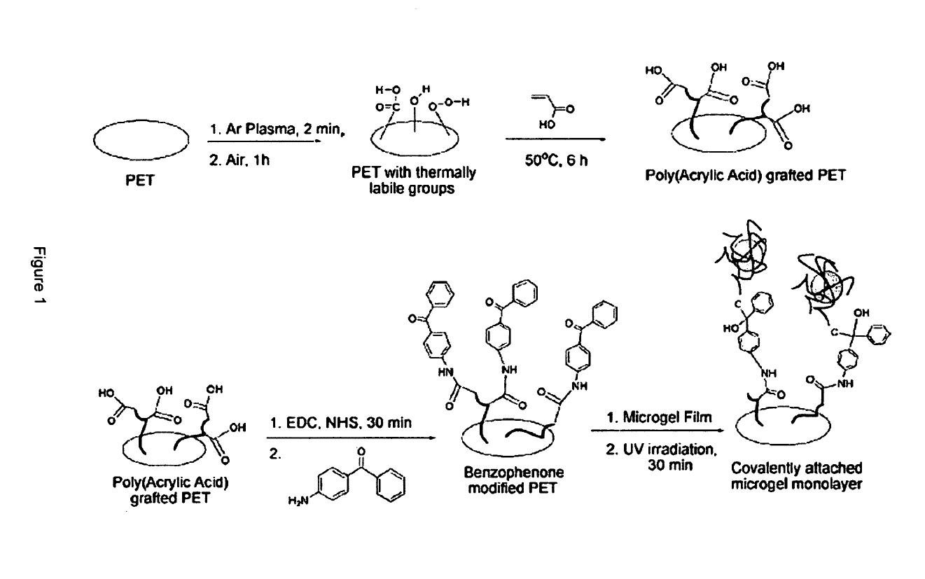

An exemplary embodiment for the functionalization of a biomaterial is illustrated in FIG. 1. In an exemplary embodiment of the present invention, chemical activation of a biomaterial can be achieved by plasma treatment (e.g., argon plasma), ozone treatment, or the like. Oxygen treatment of the chemically activated surface generates surface-active hydroperoxide species that can be used for the chemical grafting of desired chemical and biological functional groups. The chemically-activated biomaterial can be functionalized using many methods know in the art. In one embodiment of the present invention, functionalization of the activated biomaterial can comprise a linking moiety. In another embodiment of the present invention, functionalization of the activated biomaterial can comprise a plurality of linking moieties. A person of ordinary skill in the art would realize that the linking moiety used depends largely on the chemical composition of the biomaterial as well as chemical composition of the polymer microparticles to be covalently bonded to the functionalized surface of the biomaterial. In one embodiment of the present invention, a thin layer of a hydrophilic monomer (e.g., poly(acrylic acid)) can be grafted onto at least a portion of the surface of the biomaterial. The monomer can then be further modified through the use of photoaffinity labeling compounds, such as benzophenones, aryl azide, and diazirines, among others. In an embodiment of the present invention, the linking moiety can include, but it not limited to, aspects of silane chemistry, aspects of amine chemistry, aspects of bioconjugation techniques, aspects of thiol chemistry, aspects of maleimide chemistry, alkyne+azide 3+2 dipolar cycloaddition, Staudinger ligation, aspects of aldehyde chemistry, glutaraldehyde crosslinking, aspects of alcohol chemistry, or combinations thereof, among others.

In an exemplary embodiment of the present invention, surface activated PET can be functionalized by grafting a thin layer of poly(acrylic acid), and the poly(acrylic acid) modified PET is further modified by 4-aminobenzophenone (ABP) using carbodiimide coupling. In such embodiments, the PET surface is then rendered photoreactive, which can be subsequently photo-cross-linked to form a very robust interface.

Covalently bonding a plurality of cross-linked polymer microparticles to at least a portion of the functionalized surface of the biomaterial comprises can comprise disposing a plurality of cross-linked polymer microparticles onto at least a portion of the functionalized surface of the biomaterial. A plurality of cross-linked polymer microparticles can be disposed onto at least a portion of the functionalized surface of the biomaterial by many methods known in the art including, but not limited to, spin coating, dip coating, drop casting, evaporative deposition, centrifugal deposition, and the like. In some embodiments of the present invention, disposing a plurality of cross-linked polymer microparticles onto at least a portion of the functionalized surface of the biomaterial may be sufficient to covalently bond a plurality of cross-linked polymer microparticles to at least a portion of the functionalized surface. In other embodiments of the present invention, covalently bonding a plurality of cross-linked polymer microparticles to at least a portion of the functionalized surface of the biomaterial may comprise irradiation with ultraviolet (UV) light.

An aspect of the present invention comprises a method for making a coated biomaterial comprising: providing a biomaterial having a surface; functionalizing at least a portion of the surface of the biomaterial; covalently bonding a plurality of cross-linked polymer microparticles to at least a portion of the functionalized surface of the biomaterial to form a coated biomaterial; and exposing the coated biomaterial to a bio-environment, wherein an uncoated biomaterial elicits a first bio-response when placed in the bio-environment, and the coated biomaterial comprising the non-fouling polymer film elicits a second bio-response that is different than the first bio-response when placed in the bio-environment

Exposing the coated biomaterial to a bio-environment can comprise exposing the coated biomaterial to many biologically-based environments, including both in vitro and in vivo environments, capable of providing a bio-response. The methods of the present invention contemplate exposing the coated biomaterial to in vitro environments, including but not limited to cell culture (e.g., eukaryotic, prokaryotic), a medium comprising an active agent, a bioreactor, a tissue culture, an organ culture, or the like. The methods of the present invention also contemplate exposing the coated biomaterial to in vivo environments, including but not limited to humans; other animals, for example a mammal (e.g., a cow, a dog, a primate, a mouse, a rabbit, a pig, or a rat, a guinea pig), a bird, a fish, or an amphibian; or plants. Exposing the coated biomaterial to an in vivo environment can comprise providing the coated biomaterial to an in vivo environment by many known methods of implantation, embedding, contacting, and the like. As such, the coated biomaterials can be implanted in many of the same in vivo sites suitable for an appropriate medical device, as many medical devices can be coated with the non-fouling polymer film of the present invention.

An aspect of the present invention comprises a method for altering a bio-response comprising: providing a coated biomaterial comprising a non-fouling polymer film attached to at least a portion of a surface of the biomaterial; exposing the coated biomaterial to a bio-environment; and eliciting a bio-response to the coated biomaterial, wherein an uncoated biomaterial elicits a first bio-response when placed in the bio-environment, and the coated biomaterial comprising the non-fouling polymer film elicits a second bio-response that is different than the first bio-response when placed in the bio-environment. A bio-response can comprise many biological responses, activities, functions, or processes including, but not limited to adsorption of proteins and other biomolecules, cell adhesion, leukocyte activation, intracellular signaling, intercellular signaling, cytokine secretion, chemokine secretion, complement activation, inflammatory responses, production and/or release of pro-inflammatory effector molecule (e.g., reactive oxygen and nitrogen intermediates), fibrous encapsulation, receptor-ligand interactions, antigen-antibody interactions, cellular proliferation, cellular apoptosis, and cellular differentiation, among others.

In an exemplary embodiment, a method for altering a bio-response can comprise an uncoated biomaterial eliciting an inflammatory response when placed in a bio-environment, and the coated biomaterial comprising the non-fouling polymer film elicits a reduced or substantially reduced inflammatory response when placed a similar bio-environment. A reduced inflammatory response can be characterized by a reduction in biomolecule (e.g., protein) adsorption, decreased leukocyte recruitment, reduced adhesion of leukocytes, reduced activation of leukocytes, decreased expression and release of pro-inflammatory cytokines, increased expression and release of anti-inflammatory cytokines, a reduction of macrophage fusion into multi-nucleated foreign body giant cells, and limited tissue remodeling and fibrous encapsulation, among others. As used herein, the term “leukocyte” refers to the cells of the adaptive and innate immune system including, but not limited to, B lymphocytes, T lymphocytes, other lymphocytes (e.g., NK cells), neutrophils, eosinophils, basophils, monocytes, mast cells, macrophages, and other antigen presentation cells (e.g., dendritic cells).

In an embodiment of the present invention, the coated biomaterial comprising the non-fouling polymer film can elicit a reduced amount of leukocyte adhesion as compared to an uncoated biomaterial. In an embodiment of the present invention, the coated biomaterial comprising the non-fouling polymer film can adhere at least about 100% fewer leukocytes than an uncoated biomaterial. In another embodiment of the present invention, the coated biomaterial comprising the non-fouling polymer film can adhere at least about 200% fewer leukocytes than an uncoated biomaterial. In yet another embodiment of the present invention, the coated biomaterial comprising the non-fouling polymer film can adhere at least about 400% fewer leukocytes than an uncoated biomaterial. In still another embodiment of the present invention, the coated biomaterial comprising the non-fouling polymer film can adhere at least about 500% fewer leukocytes than an uncoated biomaterial.

In an embodiment of the present invention, the coated biomaterial comprising the non-fouling polymer film can elicit a reduced amount of pro-inflammatory cytokine expression adhesions as compared to an uncoated biomaterial. In an embodiment of the present invention, the coated biomaterial comprising the non-fouling polymer film can reduce pro-inflammatory cytokine expression by at least about 10%. In another embodiment of the present invention, the coated biomaterial comprising the non-fouling polymer film can reduce pro-inflammatory cytokine expression by at least about 25%. In another embodiment of the present invention, the coated biomaterial comprising the non-fouling polymer film can reduce pro-inflammatory cytokine expression by at least about 50%. In yet another embodiment of the present invention, the coated biomaterial comprising the non-fouling polymer film can reduce pro-inflammatory cytokine expression by at least about 75%. In still another embodiment of the present invention, the coated biomaterial comprising the non-fouling polymer film can reduce pro-inflammatory cytokine expression by at least about 100%.

An aspect of the present invention comprises a biomaterial comprising a non-fouling polymer film attached to at least a portion of a surface of the biomaterial, the non-fouling polymer film comprising an active agent and plurality of a cross-linked polymer microparticles, wherein at least a portion of the cross-linked polymer microparticles are covalently bonded to at least a portion of the surface of the biomaterial.

As used herein, the term “active agent” can include, without limitation, agents for gene therapy, analgesics, antiarthritics, antiasthmatic agents, anticholinergics, anticonvulsants, antidepressants, antidiabetic agents, antidiarrheals, anesthetics, antibiotics, antigens, antihistamines, anti-infectives, anti-inflammatory agents, antimicrobial agents, antimigraine preparations, antinauseants, antineoplastics, antiparkinsonism drugs, antipruritics, antipsychotics, antipyretics, antispasmodics, anorexics, antihelminthics, antiviral agents, nucleic acids, DNA, RNA, polynucleotides, nucleosides, nucleotides, amino acids, peptides, proteins, carbohydrates, lectins, lipids, fats, fatty acids, viruses, antigens, immunogens, antibodies and fragments thereof, sera, immune stimulants, immune suppressors, sympathomimetics, xanthine derivatives, cardiovascular agents, potassium channel blockers, calcium channel blockers, beta-blockers, alpha-blockers, antiarrhythmics, antihypertensives, diuretics, antidiuretics, vasodilators comprising general, coronary, peripheral, or cerebral, central nervous system stimulants, vasoconstrictors, gases, growth factors, growth inhibitors, hormones, estradiol, steroids, progesterone and derivatives thereof, testosterone and derivatives thereof, corticosteroids, angiogenic agents, antiangeogenic agents, hypnotics, immunosuppressives, muscle relaxants, parasympatholytics, psychostimulants, sedatives, tranquilizers, ionized and non-ionized active agents, anti-fungal agents, metals, small molecules, pharmaceuticals, hemotherapeutic agents, herbicides, fertilizers, wound healing agents, indicators of change in the bio-environment, enzymes, nutrients, vitamins, minerals, coagulation factors, neurochemicals, cellular receptors, radioactive materials, cells, chemical or biological materials or compounds that induce a desired biological or pharmacological effect; and combinations thereof.

In another embodiment, the an active agent may comprise proteins that may be useful in the treatment of wounds including, but not limited to, collagen, cross-linked collagen, fibronectin, laminin, elastin, and cross-linked elastin, or combinations and fragments thereof. In yet another embodiment, the matrix of the present invention may comprise acid mucopolysaccharides including, without limitation, heparin, heparan sulfate, heparinoids, dermatan sulfate, chondroitin sulfate, hyaluronic acid, cellulose, agarose, chitin, and dextran. In addition, adjuvants or compositions that enhance an immune response, as well as antibodies or antibody fragments, may also be used in conjunction with the active agents of the present invention.

In one embodiment, the matrix of the present invention may comprise a plurality of growth factor agents, which include, without limitation, basic fibroblast growth factor (bFGF), acidic fibroblast growth factor (aFGF), nerve growth factor (NGF), epidermal growth factor (EGF), insulin-like growth factors 1 and 2, (IGF-1 and IGF-2), platelet derived growth factor (PDGF), tumor angiogenesis factor (TAF), vascular endothelial growth factor (VEGF), corticotropin releasing factor (CRF), transforming growth factors α and β (TGF-α and TGF-β), granulocyte-macrophage colony stimulating factor (GM-CSF), the interleukins (e.g., interleukin-8), and the interferons.

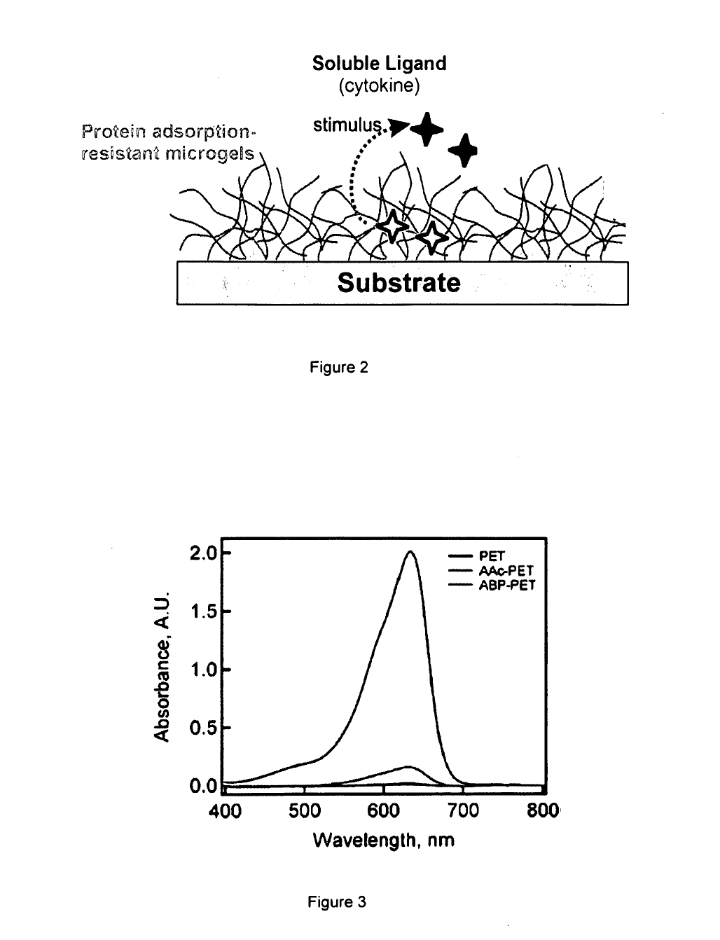

Various embodiments of the present invention comprise non-fouling polymer films designed to present, provide, and/or deliver an active agent to a bio-environment. As such, these non-fouling polymer films are capable of altering or modulating bio-responses (e.g., an inflammatory response). In an exemplary embodiment of the present invention, a non-fouling polymer film of the present invention can provide immunomodulatory agents to a bio-environment. More specifically, a non-fouling polymer film of the present invention can dynamically provide immunomodulatory agents to a bio-environment in response to specific stimulus. (FIG. 2). In an embodiment of the present invention, a non-fouling polymer film can provide an effective amount of an active agent to treat a bio-environment.

The non-fouling polymer film can comprise a plurality of cross-linked polymer microparticles comprising one or more active agents. A cross-linked polymer microparticle can comprise one or more active agents.

A non-fouling polymer film can comprise a plurality of cross-linked polymer microparticles, wherein a first population of microparticles comprises one or more active agents and wherein a second population of microparticles comprises one or more active agents. A non-fouling polymer film can comprise more than two populations of microparticles comprising one or more active agents. It is also within the scope of the present invention that a plurality of differentially-responsive microparticles may comprise one or more cross-linked polymer microparticles lacking an active agent.

The density, identity, and relative concentrations of each active agent can be controlled through the microgel surface assembly process. Therefore, the non-fouling polymer films of the present invention provide highly tunable, bioactive substrates, providing control over bio-environment-biomaterial interactions. By uniquely designing a plurality of differentially-responsive microparticles, comprising one or more of the active agents, diverse multi-responsive interfaces can be synthesized. Co-assembly of the particles in the desired ratios will result in a “mosaic” coating that has been designed with the appropriate combination of active agents, as well as the appropriate concentrations and surface densities of those active agents.

In some embodiments of the present invention, active agents can be displayed on the surface of the non-fouling polymer film. In other embodiments of the present invention, active agents can be passively released by the non-fouling polymer film into the bio-environment. In other embodiments, active agents can be actively delivered by the non-fouling polymer film in response to a stimulus into the bio-environment. In other embodiments of the present invention, non-fouling polymer films can be engineered to utilize various combinations of surface display, passive diffusion, and active delivery of active agents. The various embodiment of the present invention provide the ability to provide biological functionalities tailored for specific biotechnological and medical applications.

For example, in one embodiment of the present invention, a biomaterial comprising a non-fouling polymer film can comprise one or more soluble anti-inflammatory factors, including but not limited to, IL-1Ra, IL-4, IL-10, pirfenidone, glucocorticoids (e.g., dexamethasone), antibodies or fragments thereof (e.g., directed to pro-inflammatory cytokines), cellular receptors, ligands, among others. In another non-limiting example, a biomaterial comprising a non-fouling polymer film can comprise extracellular-matrix proteins (e.g., collagen, fibronectin, laminin, elastin), cell surface proteins, cell signaling molecules, and the like to yield functional biomaterials that have the ability to modulate cell adhesion, proliferation, and differentiation, thus mimicking a natural cellular environment.

In an embodiment of the present invention, a biomaterial comprising a non-fouling polymer film can to provide different active agents at different stages of a bio-response (e.g., an inflammatory cascade). For example, the inflammatory response to an implanted biomaterial is a cascade of events including thrombosis, neutrophil infiltration, monocyte/macrophage recruitment, adhesion and activation, which culminates in a foreign body reaction and fibrous encapsulation. Thus, release kinetics of anti-inflammatory agents can be tailored to direct macrophage activation, proliferation/apoptosis, fusion into foreign giant body cells, and cytokine release.

In an embodiment of the present invention, an active agent can be covalently associated with a cross-linked polymer microparticle by a stimulus responsive element, wherein the stimulus responsive element links the active agent to the polymer microparticle. As such, a stimulus can react on the stimulus responsive element to release the active agent from the cross-linked polymer microparticle. In an exemplary embodiment of the present invention, the provision of anti-inflammatory agents can be triggered by enzymes (i.e., a stimulus) released at different stages of the inflammatory cascade by including enzyme specific-cleavage sites (i.e., a stimulus responsive element) in the microgel coatings. Such enzyme include, without limitation, thrombin released during coagulation, esterases characteristic of monocytes/macrophages, and matrix metalloproteases (e.g., MMP-2 and MMP-9) characteristic of tissue remodeling. The various embodiments of the present invention contemplate the use of various biologically relevant proteases and enzymes for the directed release of an active agent. Thus, the embodiments of the present invention provide non-fouling polymer films capable of temporal control and localized delivery of active agents.

In an embodiment of the present invention, polymer microparticles can be prepared as spherical, monodispered microgels. These core microgels can be modified with the desired active agent. In an embodiment of the present invention, polymer microparticles can have a core/shell structure. The shell can have a thickness of about 5 nanometers to about 300 nanometers. In an exemplary embodiment of the present invention, a shell has a thickness of about 10 nanometers to about 20 nanometers. In one embodiment of the present invention, a core comprises a first active agent and the shell comprises a second active agent. In one embodiment of the present invention, the first and second active agents are the same. In an alternative embodiment of the present invention, the first and second active agents are different. Furthermore, the core and shell can be made of the same or different polymers. Both the core and the shell may comprise components amenable to biofunctionalization.

As discussed above, the polymer microparticles can be configured to provide active agents through display, passive diffusion, and active delivery, among others. In an exemplary embodiment of the present invention, a polymer microparticle having a core/shell structure, can comprise a core configured to provide active agents by passive diffusion, and the shell can be configured to provide active agents by display, active delivery, or combinations thereof.

An aspect of the present invention comprises a method for making a coated biomaterial comprising an active agent, the method comprising: providing a biomaterial having a surface; functionalizing at least a portion of the surface of the biomaterial; covalently bonding at least a plurality of cross-linked polymer microparticles to at least a portion of the functionalized surface of the biomaterial to form a coated biomaterial; and providing an active agent to at least a portion of the non-fouling polymer film.

Providing an active agent to at least a portion of the non-fouling polymer film may comprise different chemical processes depending upon the active agent and the method of providing the active agent. For example, active agents intended for passive diffusion may be passively loaded into the polymer microparticles. In the context of the active agents displayed on the surface of the polymer microparticles or active delivered through proteolytic cleavage, biofunctionalization of the polymer microparticles may be required. The biofunctionalization of polymer microparticles can be accomplished by many methods known in the art. The polymer microparticles can comprise a chemoligation motif. In an embodiment of the present invention, the chemoligation motif can be present at a concentration of about 0.5 mol % to about 15 mol %. In an embodiment of the present invention, the motif can be an alcohol side chain, such as that of co-monomer, N-(2-hydroxypropyl)methacrylamide (HPMA). The alcohol can be used to attach an azide, which in turn can be used for attachment and tethering of an active agent using ‘click’ chemistry (e.g., a Cu(I) catalyzed 3+2 dipolar cycloaddition) and Schiff base transformation, and combinations thereof. Other methods of making polymers that can do click chemistry include, but are not limited to, direct co-polymerization of an alkyne-containing comonomer and azidolysis of glycidyl methacrylate containing polymers or monomers, among others.

In some embodiments of the present invention, a protease-specific cleavage sequence can link the active agent to the polymer microparticle. Therefore, upon cleavage of protease-specific cleavage sequence by the appropriate protease, the active agent will be released from the polymer film.

An aspect of the present invention comprises a method for treating a bio-environment comprising: providing a coated biomaterial comprising a non-fouling polymer film attached to at least a portion of a surface of the biomaterial, the non-fouling polymer film comprising an active agent; exposing the coated biomaterial to a bio-environment; and providing an active agent from the coated biomaterial to the bio-environment. In an embodiment of the present invention, providing an active agent from the coated biomaterial to the bio-environment comprises providing an effective amount of an active agent from the coated biomaterial to the bio-environment to treat the bio-environment, a bio-response, or combinations thereof.

In some embodiments of the present invention, providing an active agent from the coated biomaterial to the bio-environment comprises displaying an active agent on the surface of the non-fouling polymer film. In other embodiments of the present invention, providing an active agent from the coated biomaterial to the bio-environment comprises passively releasing an active agent from the coated biomaterial to the bio-environment. In other embodiments of the present invention, providing an active agent from the coated biomaterial to the bio-environment comprises active can comprise actively delivering of the active agents by the non-fouling polymer film in response to a stimulus into the bio-environment. (FIG. 2). In still other embodiments of the present invention, providing an active agent from the coated biomaterial to the bio-environment comprises various combinations of displaying an active agent on the surface of the non-fouling polymer film, passively releasing an active agent from the coated biomaterial to the bio-environment, and actively delivering of the active agents by the non-fouling polymer film in response to a stimulus into the bio-environment.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented.

It must be noted that, as used in this specification and the appended claims, the singular forms “a,” “an,” and “the” include plural referents unless the context clearly dictates otherwise. All patents, patent applications, and references included herein are specifically incorporated by reference in their entireties.

It should be understood, of course, that the foregoing relates only to exemplary embodiments of the present invention and that numerous modifications or alterations may be made therein without departing from the spirit and the scope of the invention as set forth in this disclosure. Although the exemplary embodiments of the present invention are provided herein, the present invention is not limited to these embodiments. There are numerous modifications or alterations that may suggest themselves to those skilled in the art.

The present invention is further illustrated by way of the examples contained herein, which are provided for clarity of understanding. The exemplary embodiments should not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

Therefore, while embodiments of this invention have been described in detail with particular reference to exemplary embodiments, those skilled in the art will understand that variations and modifications can be effected within the scope of the invention as defined in the appended claims. Accordingly, the scope of the various embodiments of the present invention should not be limited to the above discussed embodiments, and should only be defined by the following claims and all equivalents.

### Example

### Example 1

### Covalent Tethering of Functional Microgel Films on Poly(Ethylene Terephthalate) Surfaces

Materials. All materials were obtained from Sigma Aldrich unless otherwise specified. The monomer NIPAm was recrystallized from hexane obtained from J. T. Baker before use. Poly(ethylene terephthalate) (PET) sheets were obtained from AIN Plastics, Marietta, Ga. All other chemicals were used as received. Formate buffer solution (pH=3.47, 10 mM) was prepared from formic acid and NaCl obtained from Fisher Scientific. Poly(ethylene glycol)diacrylate (PEG) (PEG MW 575, Polysciences, Inc.) was used as received. 1-Ethyl-3-(3-dimethylaminopropyl)carbodiimide (EDC) was purchased from Pierce. Dimethyl sulfoxide (DMSO) was obtained from J. T. Baker. Phosphate buffered saline (PBS) solution (pH 7.4, 10 mM) was prepared from NaCl (Fisher), Na₂HPO₄ (EM Science), and KH₂PO₄. Water was distilled and then purified using a Barnstead E-Pure system to a resistance of 18 MΩ and finally filtered through 0.2 μm membrane filter (Pall Gelman Metricel) before use.

Methods. Microgel Synthesis. Poly(N-isopropylacrylamide) (pNIPAm) microgel particles (100 mM total monomer concentration) were synthesized with 2 mol % poly(ethylene glycol) (PEG) diacrylate (MW 575) by a free radical precipitation polymerization method. For incorporating functional groups that can be later modified, the microgel particles were synthesized with 10 mol % acrylic acid as a co-monomer. Briefly, 0.4979 g of NIPAm monomer, 0.7011 g of cross-linker PEG-diacrylate, and 0.0025 g of surfactant sodium dodecyl sulfate (SDS) were dissolved in 49 mL of distilled, deionized (DI) water and filtered through a 0.2 μm filter. The solution was transferred to and stirred in a three-neck, round-bottom flask and heated to 70° C. while purging with N₂ gas. After reaching 70° C. and purging for 1 h, 34.3 μL of acrylic acid was added, followed by the addition of 0.0114 g (dissolved in 1 mL of DI water) of ammonium persulfate (APS) to initiate the reaction. The reaction was kept at 70° C. for 4 h. The synthesized microgels were then filtered and cleaned by five cycles of centrifugation at 15 422 g for 45 min. The supernatant was removed, and the particles were redispersed in DI water. The particles were then lyophilized overnight before being used for deposition onto the PET films.

PET Film Functionalization. PET sheets were cut into 8 mm diameter disks using biopsy punches and briefly rinsed in 70% ethanol to remove contaminants introduced during the manufacturing process. Graft polymerization of acrylic acid (AAc) on 8 mm PET films was done in two steps. PET films were first placed in a 18 W RF Ar plasma (Harrick Scientific) connected to a vacuum pump (5×10⁻⁴ mbar) for 2 min. Immediately after the Ar treatment, air was introduced into the plasma chamber and maintained at atmospheric pressure for 1 h to generate peroxide and other oxygen-containing functional groups on the PET surface. The films were immediately transferred to a round-bottom flask containing an N₂ purged 25% (v/v) aqueous solution of acrylic acid. The grafting reaction was carried out for 6 h at 50° C., after which the films were washed in water overnight. The degree of polymer grafting and hence the density of carboxyl groups on the PET surface can be controlled by varying the AAc concentration and reaction time. The pAAc modified PET was further modified with 4-aminobenzophenone (ABP) using carbodiimide coupling. The coupling of 4-aminobenzophenone is done traditionally as a one-step reaction using N,N′-dicyclohexylcarbodiimide (DCC) in organic media (DMSO). However, an aqueous carbodiimide coupling strategy was used based on activation of carboxyl groups with N-hydroxysuccinimide (NHS) and 1-ethyl-3-(3-dimethylaminopropyl)carbodiimide (EDC) and further reaction with the ABP. This is to avoid the formation of urea precipitate (the byproduct in the DCC reaction), which is difficult to remove completely from the surface being modified. The pAAc modified PET films were first activated by incubation in 2 mM EDC and 5 mM NHS in 10 mM 2-[N-morpholino]ethanesulfonic acid (MES) buffer solution (pH 6.0) for 30 min at room temperature. The films were then placed in 20 mM 2-mercaptoethanol solution in DI water to quench the EDC. The activated films were then reacted with ABP in DMSO for 2 h at room temperature. The ABP modified films were washed in DMSO and immersed in 10 mM hydroxylamine solution to quench the reaction. Finally, the films were washed in DI water.

Carboxyl Group Determination. The amount of pAAc grafting on the PET film surface was characterized by a colorimetric method based on Toluidine Blue O staining. Briefly, the grafted film was placed for 6 h at 30° C. in a 0.5 mM Toluidine Blue O solution prepared at pH 10. The film was then removed and thoroughly washed with NaOH (pH 10) to remove any dye nonspecifically adhered to the surface. The bound dye molecules were then desorbed from the film in a 50% acetic acid solution. The final dye content was determined from the optical density (OD) of the solution at 633 nm using a Shimadzu 1601 UV-visible spectrophotometer.

Particle Deposition. A spin-coating process was used to deposit a layer of microgel particles onto the functionalized PET films. The PET film was placed onto a glass slide, and the slide was placed onto the spin coater (Specialty Coating Systems) chuck and held in place by vacuum. The rotor speed was maintained at 500 rpm. Dried microgels were dispersed in a 10 mM formate buffer (pH 3.47) solution and one drop of the microgel solution was deposited onto the PET film while spinning After keeping the film on the spin coater for 100 s, a second drop of the microgel solution was deposited. The PET film was left on the spin coater for additional 100 s, and the film was allowed to dry. Finally, another drop of microgel solution was deposited on the PET by the same process, and the film was dried after 100 s of spinning This process was done on both sides of the PET films under dark conditions. Each side of the PET, with the dried microgel film, was irradiated by a 100 W longwave UV lamp (Blak-Ray) for 30 min to covalently attach the microgels onto the PET surface. The microgel-modified PET film was soaked in 10 mM phosphate buffer solution (pH 7.4) for 6 h and then washed with DI water.

Atomic Force Microscopy. All images were obtained in AC mode on an Asylum Research MFP-3D atomic force microscope (AFM). Spring constants were calculated using the thermal method. Imaging and analysis was performed using the Asylum Research MFP-3D software (written in the IgorPro environment, WaveMetrics, Inc., Lake Oswego, Or.). An Olympus AC160 cantilever with k=42 N/m, f₀=300 kHz was used for imaging.

In Vitro Cell Adhesion. The IC-21 murine macrophage cell line (ATCC; Manassas, Va.) was used to determine the bioresistant properties of the microgel coated PET in vitro. Cells were seeded at a density of 67 000 cells/cm2 on unmodified PET and microgel-coated PET disks in 24-well tissue culture-treated polystyrene plates in culture media containing 10% fetal bovine serum. After 48 h, adherent cells were fluorescently stained with calcein-AM (Molecular Probes, Eugene, Oreg.) and imaged using a Nikon TE-300 microscope to determine relative cell numbers and cell spreading on each surface.

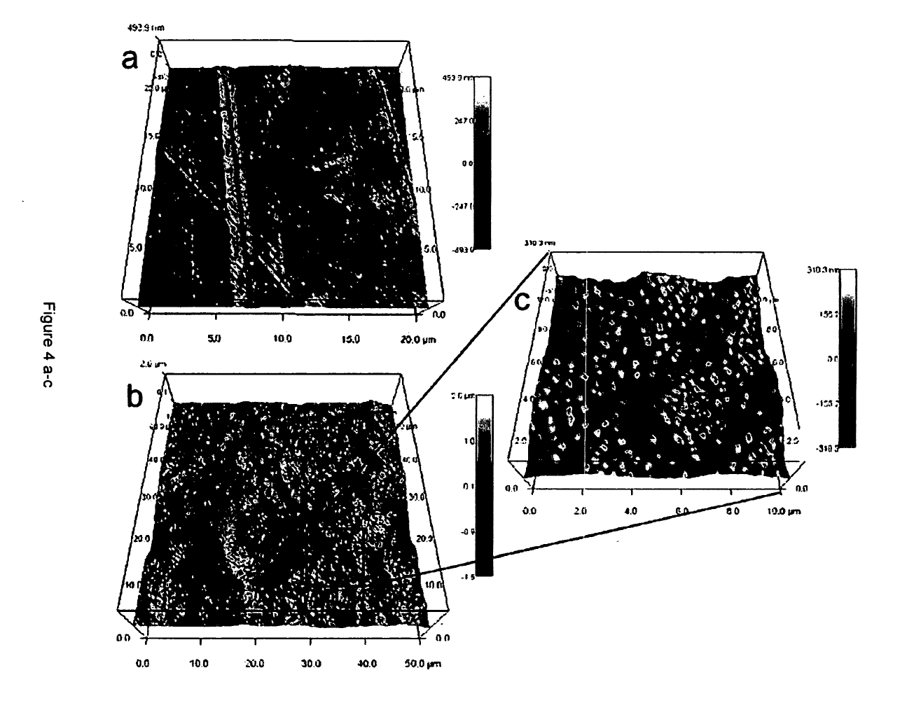

Results and Discussion. In order to deposit uniform films of microgels, the PET films had to be rendered amenable to robust particle attachment. The approach described above (FIG. 1) involves surface activation in an Ar plasma followed by the introduction of air to introduce thermally labile groups. These thermally labile groups thermally decompose to form radicals, thus initiating the polymerization of AAc to form pAAc-grafts on the PET surface. The carboxyl groups of the pAAc on the PET surface are subsequently used in the functionalization of the surface with photoaffinity label (ABP) using carbodiimide coupling chemistry. The surface grafting density of pAAc was characterized by the Toluidine blue O dye binding assay. FIG. 3 shows UV-visible absorbance spectra of Toluidine blue O dye arising from various surface treatments. Based on previous methods, by assuming a 1:1 ratio between the dye and the carboxylic acid groups, the OD at 633 nm gives a measure of the degree of_grafting. Thus, successful pAAc grafting of the PET surface is evidenced by an increase in the OD from ˜0.01 for the bare PET substrate to about 2.02 for the modified surface. The color staining of the dyed films was very uniform across the samples, suggesting relatively uniform coating of the PET (data not shown). For the pAAc grafted PET, we estimate about 1.4×10⁻⁷ mol of carboxyl groups and following the reaction with ABP, only about 1.1×10⁻⁸ mol of carboxyl groups are left on the surface. Hence, this suggests that the benzophenone_modification of the PET results in a loss of ˜92% of the carboxyl groups due to their conversion into amide groups.

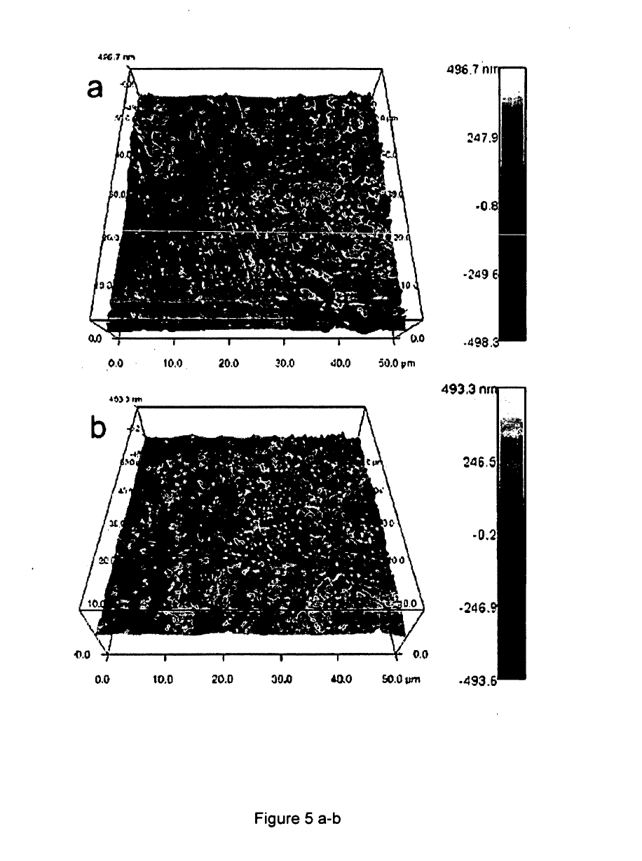

Our method of surface functionalization of the PET with photoaffinity labels results in a very efficient surface modification with the microgels. FIG. 4 shows 3D renderings of AFM images obtained from a representative film. It can be seen from the 50×50 μm scan (FIG. 4b) that there are no uncoated areas in the interrogated region. The microgels also form a dense conformal monolayer as indicated by the 10×10 μm scan (FIG. 4c). The unevenness in the microgel-coated PET is due to the uneven base surface of the PET as seen in FIG. 4a. The benzophenone modification and photocrosslinking are critically important steps for obtaining a stable monolayer, as suggested by FIG. 5. FIG. 5a shows an AFM image of a microgel film that was spin-coated onto pAAc-grafted PET without benzophenone modification, followed by extensive washing. It is clear that the coverage is sparse with only a few microgel particles retained on the surface. Since covalent linkages are not possible in the absence of the photoaffinity group, the particles cannot remain adhered to the film during the washing step. This poor coverage is probably also due, in part, to the anionic charge on both the microgels (due to the AAc co-monomers) and the film (due to the pAAc grafts). In the case of benzophenone-modified surface (FIG. 5b), slightly more microgels are retained on the PET surface, presumably due to less Coulombic repulsion between the microgels and the modified PET. In this case, the photoirradiation step is omitted, and again, no covalent attachment is possible. However, the best results are found for the pAAc-grafted PET surfaces modified by benzophenone and further photoirradiated (FIG. 5b). The photocross-linking is thus shown to provide a microgel film with excellent adhesion to the substrate and hence a presumed stability for use in biological environments. It is known that one of the key steps in the inflammatory host response to biomaterials is nonspecific protein adsorption, which then mediates cell adhesion and spreading. Recent efforts in the field of biomaterials and medical implants have focused on developing non-fouling surface treatments to prevent this nonspecific protein adsorption and cell adhesion. In addition to their nonfouling behavior, the facile and well-controlled synthesis of highly monodispersed microgels in a range of sizes, ease of their biofunctionalization using various orthogonal chemical functionalities, and possibility of co-assembling varied microgels onto a single substrate to generate complex biointerfaces makes them interesting candidates for biomedical implant coatings for modulation of inflammatory response.

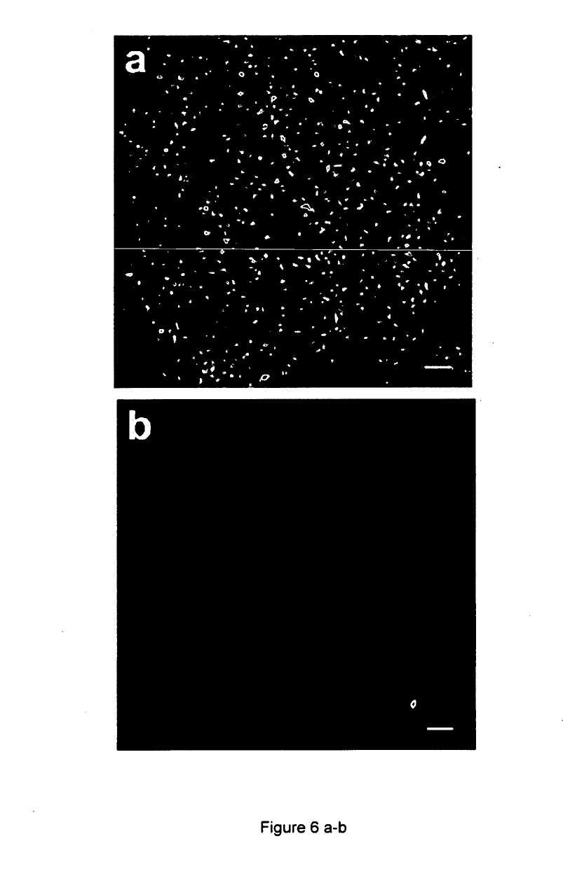

On the basis of the AFM confirmation of a stable uniform monolayer of microgels on the PET surface, the cell adhesion resistance of these surfaces was tested in vitro. IC-21 macrophages were plated on substrates in culture media containing 10% serum. This provides a rigorous test for bioresistance as cell adhesive proteins present in serum rapidly adsorb onto synthetic surfaces and mediate cell adhesion and spreading. In contrast to bare PET films, which supported high levels of cell adhesion and spreading, microgel-functionalized PET films exhibited no macrophage adhesion over the 48 h test period (FIG. 6), indicating a stable cell adhesion-resistant coating. The lack of cell adhesion to microgel-functionalized surface can be attributed to the protein-resistant nature of the PEG cross-linked microgels. The ability of microgel-coated surfaces to resist cell adhesion and spreading was distributed throughout the entire sample, indicating uniform distribution of bioresistance. The success of this surface functionalization strategy thus allows the study of the non-fouling behavior of the PEG cross-linked pNIPAm microgels in vivo and also gives us opportunities to develop more complex biomaterials incorporating multifunctional microgel monolayers.

This example provides a simple, scalable, and reproducible method of functionalizing PET with a conformal, dense film of hydrogel microparticles. The microgel layer is stable due to the covalent attachment of the microgels to the PET surface via a photoaffinity technique. This method can be easily extended for modifying the inert PET surface with any organic species, providing bioactive surfaces possessing excellent stability. Note that the spin coating deposition method is used here mainly for speed, convenience, and potential scalability. However, it may not be able to be used to coat substrates with complex geometries, and in such cases, other deposition techniques must be employed, such as dip-coating of microgels onto complex substrates.

### Example 2

### Reduced Acute Inflammatory Responses to Microgel Conformal Coatings

Materials and Methods. Sample preparation. Thin sheets of PET (AIN Plastics/ThyssenKrupp Materials NA, Madison Heights, Mich.) were cut into disks (8 mm diameter) using a sterile biopsy punch (Miltex Inc., York, Pa.) and rinsed briefly in 70% ethanol to remove contaminants introduced during the manufacturing process. pNIPAm microgel particles (100 mM total monomer concentration) were synthesized with 2 mol % PEG diacrylate (MW 575) by a free radical precipitation polymerization method, as disclosed by Nolan et al., Phase Transition Behavior, Protein Adsorption, and Cell Adhesion Resistance of Poly(ethylene glycol) Cross-Linked Microgel Particles. Biomacromolecules 6, 2032-2039 (2005), which is hereby incorporated by reference. Particle composition was confirmed by NMR. Particle size (hydrodynamic radius) and polydispersity were 334±30 nm and 1.11+0.03, respectively. Microgels were deposited on the surface of PET disks using a spin coating process as previously described in Example 1. Particles were synthesized with 10 mol % acrylic acid as a co-monomer to incorporate functional groups for future modification. All samples were rinsed in 70% ethanol on a rocker plate for 4 days, changing the solution daily to clean the samples and remove endotoxin contaminates. Prior to use, samples were rinsed three times in sterile phosphate buffered saline (PBS) and allowed to rehydrate for at least 1 hour. Samples contained 10-fold lower levels of endotoxin than the United States Food and Drug Administration's recommended 0.5 EU/mL, as determined by the LAL chromogenic assay (Cambrex, East Rutherford, N.J.).

Biomaterial surface characterization. X-ray photoelectron spectroscopy (XPS) analysis was performed on a Surface Science SSX-100 small spot ESCA Spectrometer using monochromatized A1 K alpha X-rays, 800 μm spot size, 150 eV pass energy, and take-off angle of 55°. Atomic force microscopy (AFM) images were obtained in AC mode on an Asylum Research MFP-3D atomic force microscope. Spring constants were calculated using the thermal method. Imaging and analysis was performed using the Asylum Research MFP-3D software (written in the IgorPro environment, WaveMetrics, Inc., Lake Oswego, Oreg.). An Olympus AC160 cantilever with k=42 N/m, f₀=300 kHz was used for imaging.

Fibrinogen adsorption. Fibrinogen was selected as a model plasma protein to quantify protein adsorption onto biomaterial surfaces. The amount of surface-adsorbed protein was determined using a purified solution of radiolabeled fibrinogen diluted with unlabeled fibrinogen. Samples were incubated for 1 h in a mixture of ¹²⁵I-labeled human fibrinogen (65% purity, 95% clottable, specific activity of 0.86 μCi/μg, MP Biomedicals, Irvine, Calif.) and unlabeled human fibrinogen (65% purity, 95% clottable, Sigma-Aldrich, St. Louis, Mo.) to generate a range (2-200 μg/mL) of coating concentrations. Tri(ethylene glycol)-terminated self-assembled monolayers on gold-coated glass coverslips and unmodified glass coverslips were used as controls. Following incubation in fibrinogen solutions, samples were rinsed in PBS, incubated for 30 min in a 1% solution of heat-denatured bovine serum albumin (BSA), and rinsed in PBS to remove loosely adsorbed proteins. A Packard Cobra II gamma counter was used to measure the level of radiolabeled fibrinogen adsorbed onto the samples. After correcting for background and label dilution, the amount of protein adsorbed on each sample was calculated as the radioactive counts divided by the surface area and specific activity. We note that pilot experiments demonstrated that the albumin incubation and buffer rinses only displace a small amount (<10%) of adsorbed fibrinogen from these surfaces.

Primary human monocyte isolation and culture. Peripheral human whole blood was obtained from healthy volunteer donors at the Georgia Institute of Technology Student Health Center in accordance with an approved Institute Review Board protocol (H05012). Blood (240 mL per donor) was collected into 60-mL Luer-Lok syringes; half of the blood was used to prepare autologous serum, the other half was used for monocyte isolation. To prepare autologous human serum, the blood was centrifuged (3000 rpm, 10 min, room temperature) to pellet red blood cells. The supernatant was collected, pushing down clots manually using a sterile pipette tip, and allowing further clotting (90 min, room temperature).

Human monocytes were isolated from whole blood immediately after collection using an established method developed by Anderson's group with slight modifications, as described in McNally et al., Proc. Natl Acad Sci USA 1194;91:10119-23. Cell isolations were performed on blood from three separate donors for three independent experiments (unpooled samples) with equivalent results. Collected blood was immediately treated with sodium heparin (333 U/mL blood, Baxter Healthcare, Deerfield, Ill.) as an anticoagulant. The heparinized blood was transferred to polystyrene bottles (Corning, Corning, N.Y.), diluted 1:1 with sterile PBS without calcium/magnesium, and gently swirled to mix. Peripheral blood mononuclear cells were separated using lymphocyte separation medium (Cellgro MediaTech, Herndon, Va.) by differential gradient centrifugation (400 g, 30 min at room temperature in a Thermo Fisher centrifuge, model #5682, rotor IEC 216). The mononuclear cell layer was collected and erythrocytes lysed (155 mM ammonium chloride, 10 mM potassium bicarbonate and 0.1 mM EDTA) and washed twice with sterile PBS to remove the lysis buffer. This isolation procedure yielded >95% viable cells as determined by Trypan blue exclusion. Flow cytometric analyses indicated 50±5% monocytes (CD14+) and 46±3% T/B cells (CD14−). These yields for cell viability and monocyte fractions are consistent with previous reports.

Cells were resuspended at a concentration of 5×10⁶ cells/mL in culture media (RPMI-1640 containing 25 mM HEPES, 2 mM L-glutamine [Invitrogen], 100 U/mL penicillin/streptomycin [Cellgro] and 25% autologous human serum), plated in a volume of 10 mL onto 100-mm Primaria-treated culture plates, and incubated at 37° C. and 5% CO₂. After 2 h, non-adherent cells were removed by rinsing three times with warm media. Cells were cultured for 10 days prior to plating onto experimental/control surfaces based on previous results showing that this time period provides for sufficient macrophage maturation. Media changes occurred on days 3 and 6 of culture with media containing heat-inactivated autologous serum (56° C., 1 h). By day 10 in culture, this procedure yielded 61±18% macrophages (CD64+) and 29±18% lymphocytes. The purity of macrophages increases with time in culture as non-adherent lymphocytes are washed away. We note there is evidence that lymphocytes modulate monocyte activities on biomaterials, suggesting that it is relevant to include this lymphocyte population in culture.

In vitro murine and human macrophage adhesion. Murine IC-21 macrophages (TIB-186, ATCC, Manassas, Va.) were plated at a density of 67,000 cells/cm² on unmodified PET controls and microgel-coated samples. IC-21 cells were maintained in RPMI-1640 containing 25 mM HEPES, 2 mM L-glutamine, 100 U/mL penicillin/streptomycin and 10% fetal bovine serum at 37° C. and 5% CO₂. Human monocytes were plated at 50,000 cells/cm² on microgel-coated PET or unmodified PET controls and maintained in culture media supplemented with 25% autologous human serum at 37° C. and 5% CO₂. Following 48 h of culture, biomaterial samples were rinsed three times with sterile PBS to remove loosely adherent cells. Remaining adherent cells were stained with calcein-AM (live cells) and ethidium homodimer-1 (dead cells) (Invitrogen) and imaged using a Nikon E-400 microscope equipped with epifluorescence optics and image analysis. Five representative fields per sample (4-5 independent samples per condition) were acquired (10× Plan Fluor Nikon objective, 0.30 NA), and image analysis software (ImagePro, Media Cybernetics, Silver Spring, Md.) with in-house macros was used to count adherent cells.

Murine intraperitoneal implantation. An established intraperitoneal implantation model was used to assess acute inflammatory responses. Animal procedures were conducted in accordance with an IACUC-approved protocol. Male 10-14 wk old C57BL/6 mice (Charles River Laboratories, Wilmington, Mass.) were anesthetized by isofluorane. Following a midline incision into the peritoneal cavity, sterile samples (two disks per mouse) were implanted for 48 h. Sham surgeries were performed on additional mice to be used as controls. Prior to explantation, the IP cavity was injected with 3 mL of sterile PBS containing sodium heparin (Baxter Healthcare, Deerfield, Ill.) as an anticoagulant. The abdomen was then massaged briefly, the IP lavage fluid was collected using a syringe, and disks were retrieved for analysis. One disk was used for immunofluorescence staining of adherent cells, and the second disk was used to harvest adherent cells for flow cytometric analysis of intracellular cytokine levels. Animals were sacrificed using a CO₂ chamber.

Immunofluorescence staining of adherent cells. Following careful explantation from the intraperitoneal cavity, biomaterial disks were stored briefly in PBS until completion of the retrieval surgery. Samples were then rinsed three times in PBS and fixed with 10% neutral buffered formalin. Adherent cells were permeabilized using 0.1% Triton-X 100 in PBS. Fetal bovine serum (5%) in PBS was used to block non-specific protein binding. Explants were then incubated at room temperature with a primary monoclonal antibody against the macrophage marker CD68 at a 1:200 dilution (clone KP1 from Abcam, Cambridge, Mass.). After rinsing to remove excess antibody, explants were incubated in AlexaFluor 488-conjugated goat anti-mouse IgG antibody (1:200 dilution) and counterstained with rhodamine-phalloidin (1:100 dilution) and Hoechst (1:10,000 dilution) to stain actin filaments and nuclei, respectively. Isotype control antibodies and additional staining controls demonstrated specific staining of target epitopes with minimal background. Antibodies were diluted in a solution of 1% heat-denatured BSA in PBS, and all reagents were used at 4° C. Samples were then rinsed five times in PBS and once in deionized H₂O, mounted on glass slides with coverslips, and stored in the dark at 4° C. until imaged. Eight fields per sample were acquired (20× Plan Fluor Nikon objective, 0.45 NA), and ImagePro software (Media Cybernetics, Silver Spring, Md.) with custom-designed macros was used to count the adherent cells. Results shown represent 5 or more animals per treatment group from a single implantation experiment.

Intracellular cytokine staining and flow cytometric analysis. The second disk explanted from the intraperitoneal cavity was used for measurements of cytokine expression in implant-associated cells via flow cytometry. Explanted samples were rinsed briefly in PBS and quickly transferred to a 24-well plate, and lavage samples were centrifuged to pellet cells. Cytokine staining was performed using fluorophore-labeled antibodies according to the manufacturer's protocol (eBioscience, San Diego, Calif.). Briefly, 1.0 mL of warm brefeldin A solution (3 μg/mL) in serum-containing media was added to each sample (disk or lavage fluid) to inhibit protein secretion into the media, and cells were incubated for 4 h at 37° C. to allow for cytokine accumulation within the cells.

Pilot experiments with different dissociation conditions were performed to identify protocols to efficiently isolate implant-associated cells with minimal cellular debris and appropriate staining and instrument settings for flow cytometry analysis. For cell harvest, samples were rinsed three times in cold PBS without calcium/magnesium. Disk-adherent cells were removed using warm trypsin (0.05% containing 0.53 mM EDTA), transferred to microcentrifuge tubes, and centrifuged at 300 g. The resultant cell pellet was resuspended in 1.0 mL of 10% neutral buffered formalin, and tubes were shaken at low speed on a vortexer for 10 min. A series of rinse-and-centrifuge cycles were used to remove excess fixative, and cell pellets were resuspended in a combined permeabilization/blocking buffer and replaced on the vortexer for 20 min. Fluorophore-conjugated antibodies (APC-conjugated anti-mouse TNF-α [clone MP6-XT22], FITC-labeled anti-mouse IL-1β polyclonal antibody, PE anti-mouse MCP-1 [clone 2H5], FITC-labeled anti-mouse IL-10 polyclonal antibody, eBioscience) were added to the microcentrifuge tubes at the manufacturer's recommended dilutions and shaken in the dark for 1 h. A subset of samples were stained using macrophage- and neutrophil-specific markers (PE-conjugated anti-mouse F4/80 [clone BM8] and APC-labeled anti-mouse Gr1 [clone RB6-8C5] from eBioscience and Miltenyi Biotec [Auburn, Calif.]) to label the cell populations of interest. Cells were then subjected to another series of rinse-and-centrifuge cycles to remove excess antibody and resuspended in PBS. A Becton Dickinson BD LSR digital flow cytometer was used to measure the fluorescently-labeled intracellular cytokines (counting 10,000 events per sample), and FlowJo software v7.2 (Tree Star Inc., Ashland, Oreg.) was used to analyze the data. Results shown represent 4-8 animals per treatment group from a single implantation experiment.

Statistical analysis. Data are presented as mean±standard error. Statistical analysis was performed by ANOVA using Systat 11.0 (Systat Software Inc., San Jose, Calif.). Flow cytometry histograms were compared using the Kruskal-Wallis non-parametric test. Pair-wise comparisons were performed using Tukey post-hoc tests with a 95% confidence level considered significant.

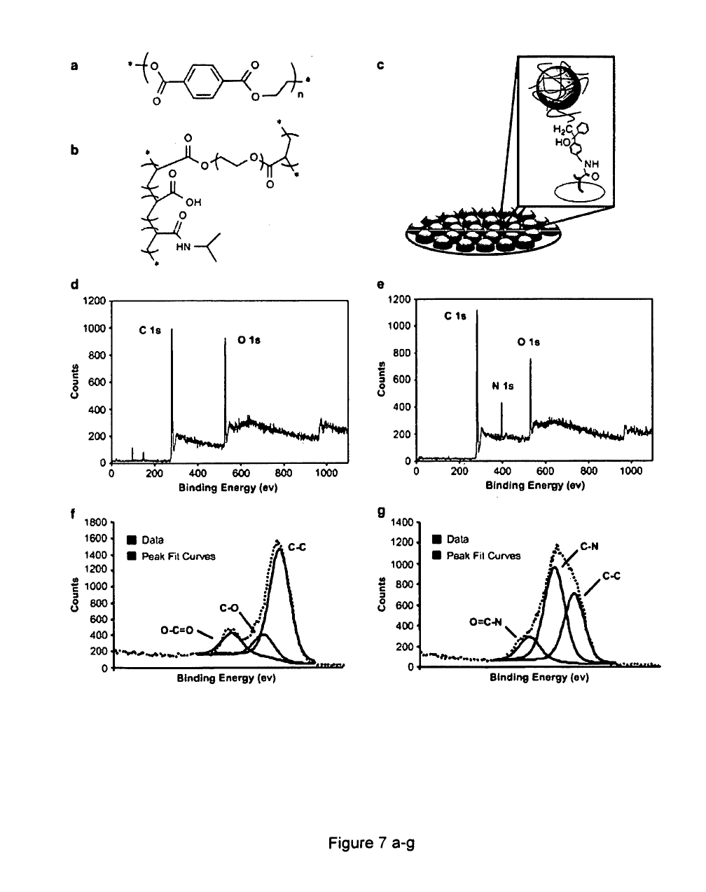

Results. Deposition of microgel particles as conformal coatings. PET substrates (FIG. 7a) were functionalized with p(NIPAM-co-AAc-co-PEGDA) microgel particles (FIGS. 7b and c), which were covalently attached to the surface via the incorporation of an aminobenzophenone photoaffinity label followed by UV excitation to form a covalently cross-linked coating (FIG. 7). Biomaterial surfaces were analyzed for both chemical composition and the uniformity of microgel deposition using XPS and AFM, respectively. XPS survey scans revealed the presence of carbon and oxygen groups on unmodified PET controls and microgel-coated surfaces (FIGS. 7d and e, respectively). Nitrogen groups (400 eV binding energy) were present only on microgel-coated surfaces (FIG. 7e). With respect to elemental composition, PET substrates contained approximately 72% carbon and 25% oxygen, whereas microgel coatings contained 77% carbon, 15% oxygen, and 9% nitrogen (all 1s orbitals). Additional high resolution scans confirmed multiple carbon bonds corresponding to the chemical structures of the PET substrate and microgel coatings (FIGS. 7f and g, respectively). In particular, there was an abundance of amide bonds characteristic of pNIPAm in the microgel coating. This chemical composition is consistent with the theoretical values.

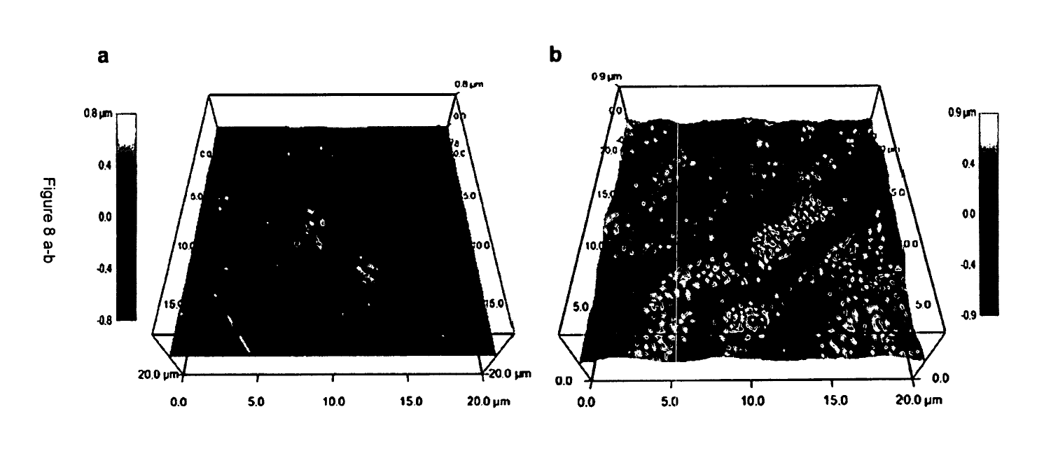

AFM images were obtained and rendered in three dimensions (FIG. 8) to visualize surface topography of the biomaterials. PET displayed a generally smooth surface (<200 nm) exhibiting scratches and surface defects (FIG. 8a), mostly likely arising from the manufacturing process. Spin coating-based deposition of the microgel particles resulted in a conformal coating on the surface with microgel particles effectively filling in scratches and covered ridges commonly present on the surface of the underlying PET substrate (FIG. 8b). The thickness of these microgel coatings is on average 160 nm (dry) and 300 nm (swollen), as determined by AFM. Comparisons between AFM analyses of substrates with incomplete and full microgel coverage indicate monolayer particle deposition, with no evidence of multilayer formation. More expansive 50×50 μm² scans also confirmed uniform microgel coverage (results not shown). The presence of these pNIPAm-specific nitrogen groups, along with AFM image analysis, confirms that the microgel particles were successfully deposited on the surface of PET disks.

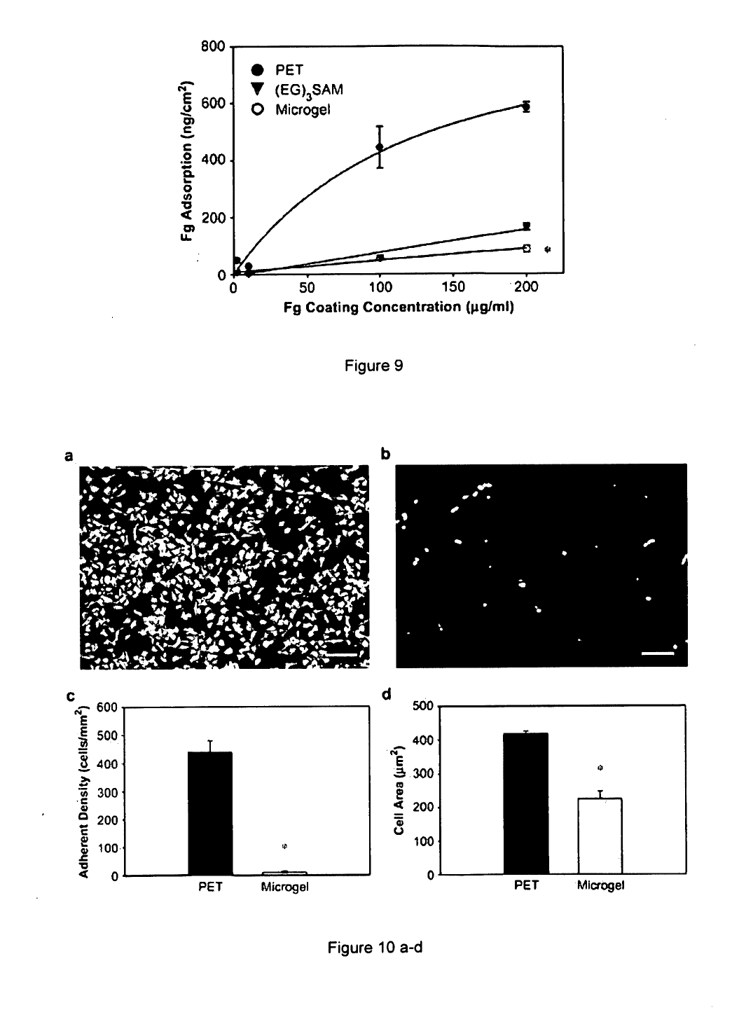

Fibrinogen adsorption studies. The ability of these microgel coatings to attenuate protein adsorption was then examined Fibrinogen was selected as the model protein for adsorption studies as this plasma component has been extensively studied in the context of host responses to synthetic materials. In addition to playing a central role in platelet adhesion to blood-contacting materials, fibrinogen adsorption promotes in vitro and in vivo leukocyte recruitment and adhesion to biomedical materials. Protein adsorption onto the surfaces was measured using ¹²⁵I-labeled human fibrinogen from a purified solution (FIG. 9). Microgel-coated samples adsorbed 7-fold lower levels of fibrinogen compared to unmodified PET disks. Additionally, the PEG-based microgel coatings performed comparably to tri(ethylene glycol)-terminated self-assembled monolayers (EG₃ SAMs) on gold-coated glass substrates, which have been extensively examined as model non-fouling surfaces. Moreover, we previously demonstrated that microgel coatings reduce albumin adsorption to background levels. Taken together, these results demonstrate that microgel-based coatings significantly reduce protein adsorption onto the underlying biomaterial substrate.

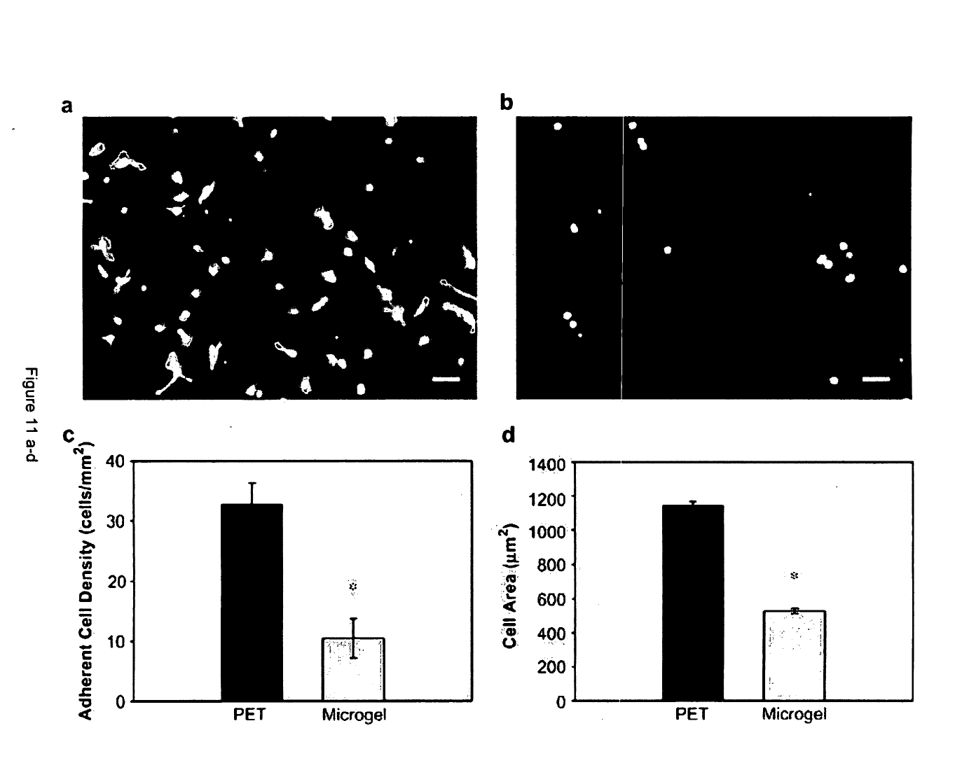

In vitro leukocyte adhesion. In vitro monocytes/macrophage adhesion to microgel-coated and unmodified PET was evaluated as a model of the leukocyte recruitment/adhesion events in the acute phase of biomaterial-induced inflammation. Murine IC-21 macrophages were plated and cultured for 48 h on biomaterials, and adherent cells were imaged and scored for viability, adherent cell density, and spread area. Unmodified PET control samples supported significant levels of cell adhesion, whereas microgel coatings exhibited 40-fold lower levels of IC-21 macrophage adhesion (FIGS. 10a and 10b, respectively), as quantified in FIG. 10c (p<1.2×10⁻⁵). Furthermore, cells adherent to unmodified PET samples had almost double the cytoplasmic spread area of those associated with microgel-coated samples (FIG. 10d, p<1.2×10⁻⁵). Calcein-AM/ethidium homodimer (Live/Dead™) staining showed >98% viability for both surfaces.

Similar studies were performed with primary human monocytes/macrophages isolated from whole blood, as these primary cells represent a more clinically relevant model. After 48 h in culture with biomaterial surfaces, adherent cells were imaged and scored for viability, adherent cell density, and spreading area. In good agreement with the murine macrophage line results, unmodified PET supported high numbers of adherent primary monocytes (FIG. 11a), whereas microgel coatings (FIG. 11b) reduced primary human monocyte/macrophage adherent cell numbers by 3-fold compared to control substrates. These results are shown graphically in FIG. 11c (p<1.1×10⁻⁴). In addition, cells adherent to unmodified PET control surfaces exhibited more cell extensions and had double the cytoplasmic spread area of those associated with microgel-coated samples (FIG. 11d, p<1.2×10⁻⁵). Calcein-AM/ethidium homodimer staining showed >95% viability for both substrates. These results demonstrate that microgel coatings significantly reduce monocyte/macrophage adhesion and spreading compared to control PET supports.

Acute inflammatory cell responses to microgel coatings. Early cellular responses to biomaterials implanted in the intraperitoneal cavity of mice were evaluated. Tang and colleagues have established this model to examine leukocyte recruitment to implanted biomaterials during the acute inflammatory process. Unmodified and microgel-coated PET disks (2 samples per mouse) were implanted for 48 h and then explanted and analyzed to determine leukocyte recruitment and adhesion as well as pro-inflammatory cytokine expression. Mice surgically treated but not receiving any biomaterial disks were used as sham controls.

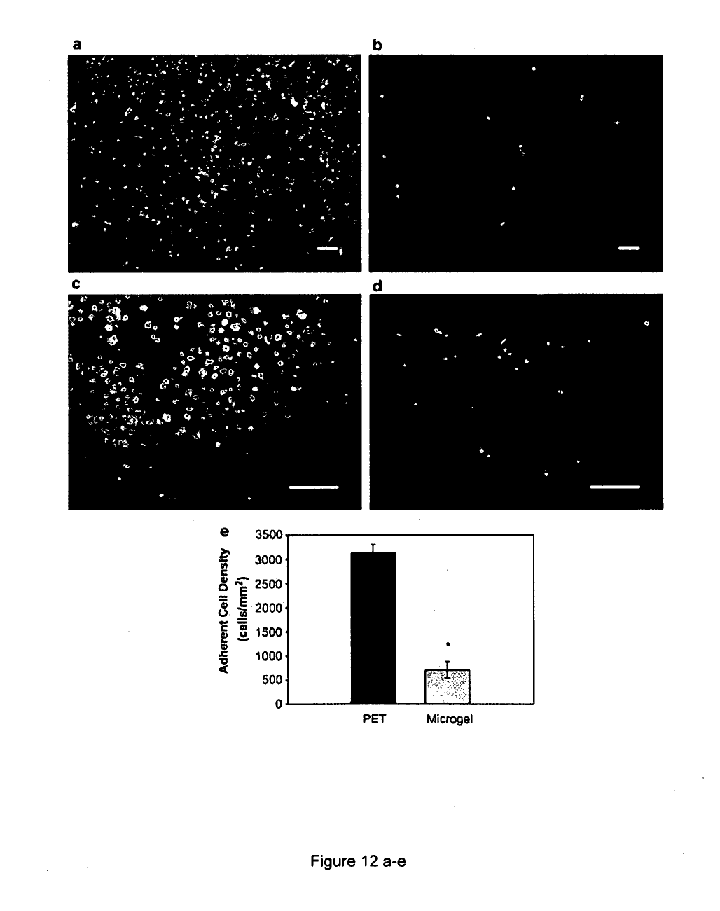

One disk explanted from each mouse was used to examine leukocyte recruitment and adhesion by cell staining and fluorescence microscopy. Following fixation and permeabilization, adherent cells were stained using an antibody against CD68 (macrophage marker), rhodamine phalloidin (actin filaments), and Hoechst (nuclei). Unmodified PET control samples displayed a dense monolayer of adherent cells (FIG. 12a). In contrast, significantly fewer cells were attached to the microgel-coated samples (FIG. 12b). Quantification of adherent cells demonstrated a 4.6-fold reduction in cell density for microgel-coated samples compared to unmodified PET (p<1.1×10⁻⁵, FIG. 12e). Furthermore, higher magnification images demonstrated fewer CD68+ macrophages on microgel-coated samples (FIG. 12d) compared to unmodified PET controls (FIG. 12c). Similar results in terms of differences in adherent cell numbers between microgel-coated and unmodified PET surfaces were observed for in a small number of samples implanted in the murine intraperitoneal space for 16 h.

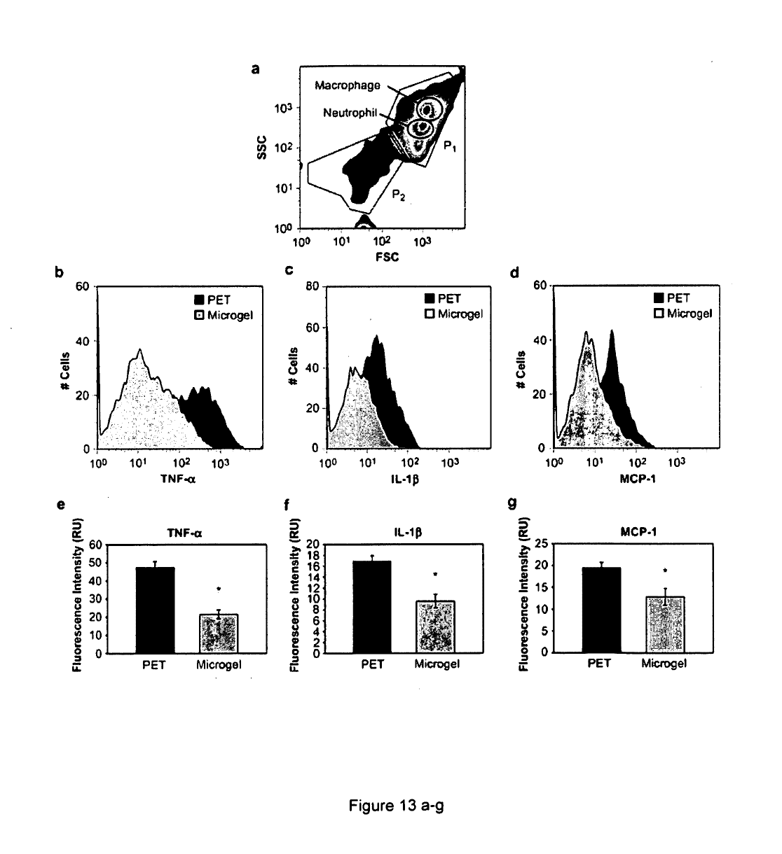

The expression of inflammatory cytokines (TNF-α, IL-1β, MCP-1, and IL-10) in implant-associated cells at 48 h of implantation was examined by flow cytometry as a measure of leukocyte activation. This cytokine profile was selected based on previous reports of acute cytokine expression around biomaterial implants. To ensure that the flow cytometry analysis was performed on whole cells and not debris for the harvest procedure, we first stained a subset of the harvested samples for markers characteristic of the cell populations, mainly macrophages and neutrophils. FIG. 13a shows a contour profile for forward scatter (FSC, proportional to particle size) vs. side scatter (SSC, proportional to antibody staining). The profile was gated for two major areas (P1, P2). The cell population in P1, which corresponds to 85% of the total number of events recorded, contains particles that (i) are large enough to represent whole cells (based on FSC values) and (ii) stain positive for macrophages and neutrophils. Therefore, analyses for cytokine expression was performed on this P1 cell population. This type of analysis is consistent with standard immunology flow cytometric analysis.

FIGS. 13b-d present histograms showing cell counts (y-axis) as a function of cytokine staining intensity (x-axis). For TNF-α, IL-1β, and MCP-1, the histograms for microgel-coated PET show a left-ward shift compared to the histograms for untreated PET. Kruskal-Wallis non-parametric tests indicated that the histograms for microgel-coated PET were statistically different from histograms for control PET (p<0.02). In addition, ANOVA of the geometric means for histograms from independent samples showed that microgel-coated samples contained significantly lower levels of pro-inflammatory TNF-α, IL-1β, and MCP-1 than unmodified PET controls (FIG. 13e-g, respectively; p<0.003). No significant differences were detected between groups for levels of anti-inflammatory IL-10 (results not shown). Additionally, a peritoneal lavage was performed to collect fluid in the tissue exudates proximal to the implant. No differences were detected between surface treatments for pro-inflammatory cytokine expression of cells in the exudate, and these levels of cytokine expression were similar to the sham controls. These results demonstrate that leukocyte activation was dependent on adhesion to the biomaterial implant. Furthermore, microgel coatings attenuate leukocyte activation and significantly reduce expression of pro-inflammatory cytokines compared to PET substrates.

The present example provides a coating strategy based on thin films of poly(N-isopropylacrylamide-co-acrylic acid) hydrogel microparticles cross-linked with PEG diacrylate. These microgel particles were spin-coated and covalently grafted onto PET substrates. XPS and AFM analyses demonstrated that these particles were deposited as dense conformal coatings. Attractive features of this coating technology include (i) precise control over particle synthesis in terms of composition and structure, (ii) ability to generate complex architectures and/or functionalities, including controlled drug release, and (iii) ability to generate ‘mosaic’ complex coatings containing variations in particle composition and/or spatial arrangement via modular assembly and soft lithography. In addition, these particles can be deposited onto different substrates by various means, including spin coating, centrifugation, and dip-coating. We note that the amount of mass attached with just a few chemical reactions at the surface is potentially extraordinarily high, which should be beneficial for obtaining high densities of PEG and good surface coverage. Compared to many ‘grafting-to’ and surface polymerization reactions, this approach provides a more controllable route. Nevertheless, generation of dense, conformal microgel coatings requires optimization of particle deposition parameters, including covalent tethering, and may not be easily applicable to surfaces with complex geometries/topographies.

In vitro protein adsorption onto microgel-coated and uncoated PET were examined using radiolabeled fibrinogen as a model plasma protein. Microgel coatings significantly reduced fibrinogen adsorption compared to unmodified PET. Additionally, the PEG-based microgel coatings performed equivalently to self-assembled monolayers presenting tri(ethylene glycol). The significant reductions in adsorbed fibrinogen for microgel coatings are in good agreement with previous results for low adsorption of serum albumin to these films. The levels of fibrinogen adsorbed onto microgel coatings (60 ng/cm² at 30 μg/mL coating concentration) are comparable to protein densities (40-60 ng/cm²) adsorbed onto PEG/PEO polymers grafted onto surfaces. However, the density of fibrinogen adsorbed onto the microgel coatings is considerably higher than adsorbed protein densities (<10 ng/cm²) onto dense brushes of oligo(ethylene glycol)methacrylate and poly(2-methacryloyloxyethyl phosphorylcholine) generated by surface-initiated polymerization reactions. Furthermore, the fibrinogen adsorption levels for the microgel coating are also higher than fibrinogen adsorption values (<10 ng/cm²) reported for glow discharge plasma-deposited tetraethylene glycol dimethyl ether densely cross-linked coatings (“tetraglyme”). The differences in protein adsorption resistance among these coating technologies probably arise from differences in the architecture/structure of the PEG chains as the chain length and grafting density strongly influence “non-fouling” behavior. An alternative explanation for the higher values of adsorbed fibrinogen to the microgel coatings is that there are spaces between microgel particles below the resolution of the AFM rendering that provide sites for protein adsorption. This potential limitation could be addressed by using a different deposition technique or multi-layers of microgel particles.

Microgel-coated PET exhibited significant reductions in in vitro cell adhesion and spreading compared to untreated PET for both an established murine macrophage cell line and primary human monocytes/macrophages. The reduced levels of cell adhesion and spreading on microgel-coated surfaces provide indirect evidence for the lack of adsorption of cell-adhesion promoting proteins. We observed high levels of viability between surface conditions so we do not attribute the differences in adherent cell numbers and spreading to differences in cell viability between the surfaces. These cell adhesion results are consistent with previous reports of very low in vitro monocytes/macrophage adhesion to PEG-functionalized materials such as tetraglyme and PEG-star coatings. In contrast, other studies showed high monocytes/macrophage adhesion to surfaces grafted with PEO polymers or PEG-containing interpenetrating networks; however, in vitro macrophage fusion into foreign body giant cells was significantly decreased on these coatings. The reason(s) for these discrepancies in monocytes/macrophage adhesion among PEG-based coatings remains poorly understood. These PEG-based coatings significantly reduce protein adsorption, albeit to different extents, and prevent adhesion of other cell types such as osteoblasts and endothelial cells. Possible explanations include (i) differences in adhesion receptor repertoire or numbers between primary monocytes/macrophages and other cell types and (ii) increased cell type-dependent degradation/modification of the underlying PEG coating.

We evaluated acute inflammatory cellular responses to microgel coatings in a murine intraperitoneal implant model. Microgel coatings significantly reduced the number of adherent leukocytes compared to uncoated PET at 48 h of implantation. Similar differences were observed in a small number of samples implanted for 16 h. These reductions in in vivo leukocyte adhesion for the microgel coatings are in good agreement with our in vitro cell adhesion findings. Furthermore, analysis of cytokine expression in adherent leukocytes demonstrated that microgel coatings reduced expression of the pro-inflammatory cytokines TNF-α, IL-1β, and MCP-1 compared to untreated microgel coatings following 48 h implantation. This analysis is based on comparing equal numbers of cells; because microgel-coated implants contained 4.6-fold fewer cells than untreated PET implants, we expect that the total cytokine load will be significantly reduced for the microgel-coated implants. Differences in cytokine expression were only detected for adherent cells and were not evident in cells isolated from lavage fluid, suggesting that adhesion to the implant was necessary for increased cytokine expression. Taken together, these results indicate that microgel coatings reduce acute inflammatory cell adhesion and cytokine expression in vivo.

Several mechanisms could explain the ability of microgel coatings to significant reduce in vivo leukocyte adhesion and cytokine expression, especially when considering that these coatings exhibited higher levels of protein adsorption compared to tetraglyme and other PEO-based films. First, the higher levels of adsorbed proteins may be due to adsorption in spaces between microgel particles that are inaccessible to cells, resulting in dense conformal coatings with respect to the cells. Alternatively, because our assembly process deposits a high volume polymer film (swollen microgel coatings are ˜300 nm thick, tetraglyme coatings are 100 nm). It is possible that the microgel coatings undergo slower overall degradation than other coatings. Finally, an intriguing possibility is that the topography, in combination with the surface chemistry, of the microgel coating reduces leukocyte adhesion.

1. A coated biomaterial capable of altering a bio-response, the biomaterial comprising a non-fouling polymer film attached to at least a portion of a surface of the biomaterial, the non-fouling polymer film comprising a plurality of a cross-linked polymer microparticles, wherein at least a portion of the cross-linked polymer microparticles are covalently bonded to at least a portion of the surface of the biomaterial, wherein an uncoated biomaterial elicits a first bio-response when placed in a bio-environment, and the coated biomaterial comprising the non-fouling polymer film elicits a second bio-response that is different than the first bio-response when placed in a similar bio-environment.

2. The coated biomaterial of claim 1, wherein the uncoated biomaterial elicits a first bio-response when placed in a bio-environment, and the coated biomaterial comprising the non-fouling polymer film elicits a second bio-response that is less than the first bio-response when placed a similar the bio-environment.

3. The coated biomaterial of claim 2, wherein the bio-environment is an in vivo system and the bio-response in an inflammatory response.

4. The coated biomaterial of claim 1, wherein the uncoated biomaterial elicits a first bio-response when placed in a bio-environment, and the coated biomaterial comprising the non-fouling polymer film elicits a second bio-response that is greater than the first bio-response when placed a similar the bio-environment.

5. The coated biomaterial of claim 4, wherein the bio-environment is an in vivo system and the bio-response in a wound healing response.

6. The coated biomaterial of claim 1, wherein the non-fouling polymer film adsorbs at least about 100% less protein than an uncoated biomaterial.

7. The coated biomaterial of claim 1, wherein the non-fouling polymer film adheres at least about 100% fewer cells than an uncoated biomaterial.

8. The coated biomaterial of claim 1, wherein the non-fouling polymer film in its solvent swollen state comprises a thickness of about 10 nanometers to about 10 micrometers.

9. The coated biomaterial of claim 1, wherein the cross-linked polymer microparticles comprises poly(N-isopropylacrylamide) cross-linked with poly(ethylene glycol)diacrylate.

10. The coated biomaterial of claim 9, wherein the poly(ethylene glycol)diacrylate has a molecular weight of less than about 575 Da and a concentration of about 2 mol %.

11-96. (canceled)

97. A method for making a coated biomaterial comprising:
providing a biomaterial having a surface;
functionalizing at least a portion of the surface of the biomaterial; and
covalently bonding a plurality of cross-linked polymer microparticles to at least a portion of the functionalized surface of the biomaterial to form a coated biomaterial;

98. The method for making a coated biomaterial of claim 97, wherein functionalizing at least a portion of the surface of the biomaterial comprises activating at least a portion of the surface of the biomaterial with a plasma, reacting the activated surface with oxygen to form a reactive species on the surface, grafting a linking moiety to the reactive species of the activated surface, and rendering the surface of the photoreactive with a photoaffinity labeling compound.

99. The method for making a coated biomaterial of claim 98, wherein covalently bonding a plurality of cross-linked polymer microparticles to at least a portion of the functionalized surface of the biomaterial to form a coated biomaterial comprises disposing a plurality of cross-linked polymer microparticles onto at least a portion of the functionalized surface of the biomaterial.

100. The method for making a coated biomaterial of claim 99, wherein covalently bonding a plurality of cross-linked polymer microparticles to at least a portion of the functionalized surface of the biomaterial to form a coated biomaterial further comprises reacting the photoreactive surface of the biomaterial with at least a portion of a plurality of cross-linked polymer microparticles in the presence of ultraviolet radiation.

101. The method for making a coated biomaterial of claim 97, further comprising providing an active agent to at least a portion of the non-fouling polymer film.

102. The method for making a coated biomaterial of claim 101, wherein providing an active agent to at least a portion of the non-fouling polymer film comprises biofunctionalization of at least a portion of the plurality of cross-linked polymer microparticles with a chemoligation motif.

103. A coated biomaterial comprising a non-fouling polymer film attached to at least a portion of a surface of the biomaterial, the non-fouling polymer film comprising an active agent and plurality of a cross-linked polymer microparticles, wherein at least a portion of the cross-linked polymer microparticles are covalently bonded to at least a portion of the surface of the biomaterial.

104. The coated biomaterial of claim 103, wherein the cross-linked polymer microparticles comprises poly(N-isopropylacrylamide) cross-linked with poly(ethylene glycol)diacrylate.

105. The coated biomaterial of claim 103, wherein the non-fouling polymer films provides an active agent to a bio-environment by display of an active agent on the surface of the non-fouling polymer film, passive diffusion of an active agent from the non-fouling polymer film, active delivery of the active agent from the non-fouling polymer film, or combinations thereof.

106. The coated biomaterial of claim 103, wherein the active agent is covalently associated with a cross-linked polymer microparticle by a stimulus responsive element, wherein a stimulus acts on the stimulus responsive element to release the active agent from the cross-linked polymer microparticle.
